(19) United States
(12) Patent Application Publication
(10) Pub. No.: US 2024/0265821 A1
(43) Pub. Date: Aug. 8, 2024

(54) Tailored Synthetic Personas with Parameterized Behaviors

(71) Applicant: CenturyLink Intellectual Property LLC, Broomfield, CO (US)

(72) Inventors: Patrick Giagnocavo, Littleton, CO (US); Michael Stillman, Pensacola, FL (US)

(73) Assignee: CenturyLink Intellectual Property LLC, Broomfield, CO (US)

(21) Appl. No.: 18/545,199

(22) Filed: Dec. 19, 2023

Related U.S. Application Data
(60) Provisional application No. 63/483,354, filed on Feb. 6, 2023.

(51) Int. Cl. G09B 5/02; G09B 5/04; G09B 5/06

(52) U.S. Cl. CPC G09B 5/02; G09B 5/04; G09B 5/06; G06F 40/58

(57) ABSTRACT

Novel tools and techniques are provided for implementing tailored synthetic personas with parameterized behaviors. In various embodiments, a computing system may cause an AI/ML-driven persona(s) to interact with a user via a UI, the interaction including a conversation between the AI/ML-driven persona(s) and the user. Using at least one AI/ML model, the computing system may analyze the conversation to identify a goal(s) of the conversation, may determine a structure of the interaction, may determine one or more first parameters for the determined structure of the interaction (the one or more first parameters defining conversational guardrails for steering the interaction away from conversational tangents), may generate one or more first conversational threads configured to achieve the goal(s) of the conversation, and may cause the AI/ML-driven persona(s) to continue the conversation with the user using the one or more first conversational threads to work toward achieving the goal(s) of the conversation.

### CROSS-REFERENCE TO RELATED APPLICATIONS

This application claims the benefit of U.S. Provisional Application No. 63/483,354 filed Feb. 6, 2023, entitled “Tailored Synthetic Personas with Parameterized Behaviors,” which is incorporated herein by reference in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to methods, systems, and apparatuses for implementing computerized human interaction systems, and, more particularly, to methods, systems, and apparatuses for implementing tailored synthetic personas with parameterized behaviors.

### BACKGROUND

In some user interactive systems where a computerized system is used to interact with a user (e.g., a interactive voice response (“IVR”) system or the like), the interactions are very limited and typically frustrating for the user to navigate. In some user interactive systems in which human agents are talking with the user, there are usually multiple different agents who are randomly selected to talk with the user, which necessitates the agents each having to catch up to the conversation and/or to ask the user to repeat themselves. In some cases, because human agents are restricted from accessing some information associated with the user (for privacy reasons, for instance), the interactions are necessarily limited in scope. In some instances, some human agents may have accents that may make conversations with some users difficult.

It is with respect to this general technical environment to which aspects of the present disclosure are directed.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components. For denoting a plurality of components, the suffixes “a” through “n” may be used, where n denotes any suitable integer number (unless it denotes the number 14, if there are components with reference numerals having suffixes “a” through “m” preceding the component with the reference numeral having a suffix “n”), and may be either the same or different from the suffix “n” for other components in the same or different figures. For example, for component #1 105a-105n, the integer value of n in 105n may be the same or different from the integer value of n in 110n for component #2 110a-110n, and so on.

FIG. 1 is a schematic diagram illustrating a system for implementing tailored synthetic personas with parameterized behaviors, in accordance with various embodiments.

FIG. 2 is a schematic diagram illustrating a non-limiting example of a system for implementing tailored synthetic personas with parameterized behaviors for engaging in a product(s) and/or service(s) interaction with a user, in accordance with various embodiments.

FIG. 3 is a schematic diagram illustrating a non-limiting example of a system for implementing tailored synthetic personas with parameterized behaviors for engaging in a skills training interaction with a user, in accordance with various embodiments.

FIG. 4 is a schematic diagram illustrating a non-limiting example of a system for implementing tailored synthetic personas with parameterized behaviors for engaging in a therapy or social skills training interaction with a user, in accordance with various embodiments.

FIGS. 5A-5I are flow diagrams illustrating a method for implementing tailored synthetic personas with parameterized behaviors, in accordance with various embodiments.

FIG. 6 is a block diagram illustrating an exemplary computer or system hardware architecture, in accordance with various embodiments.

### DETAILED DESCRIPTION OF CERTAIN EMBODIMENTS

### Overview

Various embodiments provide tools and techniques for implementing computerized human interaction systems, and, more particularly, to methods, systems, and apparatuses for implementing tailored synthetic personas with parameterized behaviors.

In various embodiments, a computing system may cause at least one AI/ML-driven persona to interact with a user via a UI, the interaction including a conversation between the at least one AI/ML-driven persona and the user. Using at least one AI/ML model among one or more AI/ML models, the computing system may analyze the conversation to identify one or more goals of the conversation, may determine a structure of the interaction with the user, may determine one or more first parameters for the determined structure of the interaction with the user, the one or more first parameters defining conversational guardrails for steering the interaction away from conversational tangents and toward achieving at least one goal of the conversation, may generate one or more first conversational threads configured to achieve the at least one goal of the conversation among the one or more goals of the conversation within the determined structure of the interaction with the user, in some cases, based on the one or more first parameters, and may cause the at least one AI/ML-driven persona to continue the conversation with the user using the one or more first conversational threads to work toward achieving the at least one goal of the conversation.

In some embodiments, the one or more goals of the conversation may be related to at least one of one or more products or one or more services provided by a provider, and the computing system may cause the at least one AI/ML-driven persona to interact with the user to engage in conversation related to at least one goal related to at least one of the one or more products or the one or more services provided by the provider.

According to some embodiments, the computing system may cause at least one AI/ML-driven persona to interact with a user via a UI, the interaction including a conversation between the at least one AI/ML-driven persona and the user during which the at least one AI/ML-driven persona engages in assisting the user in learning a skill, including, but not limited to, a skill in a language.

In some examples, the computing system may cause at least one AI/ML-driven persona to interact with a user via a UI, the interaction including a conversation between the at least one AI/ML-driven persona and the user during which the at least one AI/ML-driven persona engages in assisting the user in learning one or more social skills to interact with other people, including, but not limited to, a skill for interacting with multiple people at a time.

During the interactions, the AI/ML-driven persona(s) may be flexible such as to adapt, adjust, and/or interact with the user, in order to enhance the interaction with the user. Where humans may not have access to particular information or data associated with the user (e.g., contract with the user, historical interactions with the user, order history of the user, contact information of the user, other personal information about the user, etc.), the AI/ML-driven persona(s) has access to all available information about the user, and can seamlessly utilize said data to enhance the interaction with the user. Also, where certain human-based interactive systems use multiple different people (sometimes by random selection), which results in the user having to repeat information and to confirm details, the AI/ML-driven persona(s) (even if utilizing multiple personas) can seamlessly integrate all available information about the user as well as historical data and previous interactions to improve the interaction with the user, and in a manner that simulates a natural conversation between friends, associates, or other familiars.

These and other aspects of the tailored synthetic personas with parameterized behaviors are described in greater detail with respect to the figures.

The following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

In an aspect, a method may comprise causing, by a computing system, at least one artificial intelligence (“AI”)/machine learning (“ML”)-driven persona to interact with a user via a user interface (“UI”), the interaction including a conversation between the at least one AI/ML-driven persona and the user; analyzing, by the computing system and using one of at least one AI/ML model, the conversation to identify one or more goals of the conversation related to at least one of one or more products or one or more services provided by a provider; generating, by the computing system and using one of the at least one AI/ML model, one or more first conversational threads configured to achieve at least one goal of the conversation among the one or more goals of the conversation related to the at least one of the one or more products or the one or more services; and causing, by the computing system, the at least one AI/ML-driven persona to continue the conversation with the user using the one or more first conversational threads to work toward achieving the at least one goal of the conversation.

In some embodiments, the computing system may comprise at least one of a server, an AI system, a ML system, an AI/ML system, a deep learning (“DL”) system, a user interactive system, a customer interface server, a cloud computing system, or a distributed computing system, and/or the like. In some cases, the UI may comprise one of a voice-only UI, a telephone communication UI, a video-only UI, a video with voice UI, a chat UI, a software application (“app”) UI, a holographic UI, a virtual reality (“VR”)-based UI, an augmented reality (“AR”)-based UI, a mixed reality (“MR”)-based UI, or a web-portal-based UI, and/or the like.

According to some embodiments, the one or more goals of the conversion may comprise at least one of purchasing the one or more products, ordering the one or more services, answering one or more questions regarding at least one product among the one or more products, answering one or more questions regarding at least one service among the one or more services, learning how to use at least one product among the one or more products, learning how to use at least one service among the one or more services, troubleshooting at least one issue associated with at least one product among the one or more products, troubleshooting at least one issue associated with at least one service among the one or more services, returning the one or more products, or ending the one or more services, and/or the like.

Merely by way of example, in some cases, the one or more products may comprise one or more of a telephone, a modem, a router, a customer premises equipment (“CPE”), an Ethernet circuit, a network device, a server, a consumer product, an electronic device, sporting goods, office equipment, a home appliance, a media recording device, a media player, a user device, clothing, footwear, a vehicle, or a building, and/or the like. In some examples, the one or more services may comprise one or more of electricity utility service, water utility service, trash and recycling pickup service, telephone service, cellular phone service, satellite telephone service, digital subscriber line (“DSL”) service, Internet service, Ethernet service, optical fiber Internet service, satellite Internet service, streaming media service, downloadable media service, cable television service, or satellite television service, and/or the like.

In some embodiments, the method may further comprise, concurrent with the interaction with the user, causing, by the computing system, the at least one AI/ML-driven persona to investigate a status of the at least one of the one or more products or the one or more services; determining, by the computing system, whether the computing system is capable of remotely addressing one or more issues with the at least one of the one or more products or the one or more services; and based on a determination that the computing system is capable of remotely addressing one or more issues with the at least one of the one or more products or the one or more services, generating, by the computing system, a conversational message indicating that the at least one AI/ML-driven persona is able to remotely address the one or more issues with the at least one of the one or more products or the one or more services and will proceed to do so, and initiating, by the computing system, one or more processes to remotely address the one or more issues with the at least one of the one or more products or the one or more services. In some examples, determining whether the computing system is capable of remotely addressing the one or more issues with the at least one of the one or more products or the one or more services may comprise remotely accessing, by the computing system, one or more systems associated with the at least one of the one or more products or the one or more services, and remotely running, by the computing system, one or more tests on the one or more systems, the one or more tests including a connectivity and control test.

According to some embodiments, the method may further comprise analyzing, by the computing system and using one of the at least one AI/ML model, the interaction to identify one or more observable characteristics of the user, the one or more observable characteristics including at least one of one or more speech patterns of the user, a language used by the user, whether the user has an accent, what accent the user has, whether English is a second language for the user, one or more non-verbal cues of the user, a demeanor of the user, a sentiment of the user, or an emotional state of the user, and/or the like; accessing and analyzing, by the computing system and using one of the at least one AI/ML model, stored information associated with the user to identify one or more conversation points, the stored information including at least one of account information associated with the user, contact information associated with the user, billing information associated with the user, one or more contracts between the provider and the user, order history data associated with the user, previous interactions with the user, previous trouble tickets associated with the user, historical data associated with the user, demographic information about the user, personal information about the user, user-volunteered information regarding general interests of the user, information regarding a market segment within which the user is classified, or societal information for a societal segment to which the user belongs, and/or the like; and causing, by the computing system, the at least one AI/ML-driven persona to adapt by modifying the interaction with the user, based at least in part on at least one of the identified one or more observable characteristics of the user or the identified one or more conversation points, to enhance or improve the interaction with the user. In some instances, the method may further comprise identifying and verifying, by the computing system, an identity of the user, based at least in part on one or more of caller identification (“ID”) information, the account information associated with the user, a voiceprint of the user, two-factor authentication, or information provided by the user, and/or the like.

In some embodiments, the at least one AI/ML-driven persona may be among a plurality of AI/ML-driven personas comprising at least one of one or more personas based on a fictional literary character, one or more personas based on a non-fictional literary character, one or more personas based on a comic-book character, one or more personas based on a cartoon character, one or more personas based on an anime character, one or more personas based on a manga character, one or more personas based on a television character, one or more personas based on a movie character, one or more personas based on a character from an advertisement, one or more personas based on a mascot, one or more personas based on a meme, one or more personas based on an athlete, one or more personas based on a sports personality, one or more personas based on a news personality, one or more personas based on a political personality, one or more personas based on a reality television personality, one or more personas based on a social media influencer, one or more personas based on a living celebrity, one or more personas based on a deceased celebrity, one or more personas based on a historical figure, one or more personas based on a fictionalization of a historical figure, one or more personas based on a character played by an actor or actress, one or more personas based on a bespoke character, or one or more personas simulating average humans in a geographical area within which the user is currently located or was previously residing, and/or the like.

According to some embodiments, the UI may be a visual-based UI, wherein the method may further comprise generating, by the computing system, an avatar for each of the at least one AI/ML-driven persona; displaying, by the computing system and within the UI, the avatar for each of the at least one AI/ML-driven persona; and animating, by the computing system and within the UI, the avatar in synchronization with the conversation with the user, wherein the interaction may further comprise the animation of the avatar.

In some instances, each AI/ML-driven persona may have a set personality, the set personality including at least one of a set speech pattern, a set mannerism, a set command of one or more languages, a set accent, a set collection of non-verbal cues, or a set collection of emotional demeanors, and/or the like. In some examples, each of the interaction, the conversation, the one or more first conversational threads, and the animation of each AI/ML-driven persona may be performed in a manner consistent with the set personality of said AI/ML-driven persona.

In some cases, the method may further comprise, in response to user selection of a gamification mode, performing the following: generating, by the computing system, a visual representation of a list of the one or more goals of the conversation; and in response to achieving a goal among the one or more goals of the conversation, generating, by the computing system, an animation of one or more of the at least one AI/ML-driven persona performing one or more actions comprising checking off the achieved goal, removing the achieved goal from the list, or marking the achieved goal as having been achieved, the one or more actions being performed in a manner consistent with the set personality of each of the one or more of the at least one AI/ML-driven persona.

In some examples, the method may further comprise, prior to the conversation or during a setup phase, performing one of: receiving, by the computing system, a user selection of the at least one AI/ML-driven persona from a licensed set of AI/ML-driven personas among the plurality of AI/ML-driven personas with whom to interact; identifying, by the computing system, the user, and selecting, by the computing system, the at least one AI/ML-driven persona from the licensed set of AI/ML-driven personas to match the user for interacting with the user, based on information regarding the identified user; setting, by the computing system, a default set of AI/ML-driven personas from the licensed set of AI/ML-driven personas, wherein the default set of AI/ML-driven personas comprises the at least one AI/ML-driven persona; or randomly selecting, by the computing system, the at least one AI/ML-driven persona from the licensed set of AI/ML-driven personas for interacting with the user; or the like.

In some instances, the method may further comprise at least one of: adapting or adjusting, by the computing system and using one of the at least one AI/ML model, a personality of one or more of the at least one AI/ML-driven persona to mold to or match a determined personality of the user, wherein the personality of the user is determined based on at least one of analysis of the interaction with the user, analysis of a previous interaction with the user, or known information about the user, and/or the like; or adapting or adjusting, by the computing system and using one of the at least one AI/ML model, one or more interaction characteristics of one or more of the at least one AI/ML-driven persona to match to a determined corresponding interaction characteristic of the user, the one or more interaction characteristics including at least one of speech pattern, language, accent, cultural mannerisms, cultural phraseology, general mannerisms, general phraseology, slang, jargon, or sentiment, and/or the like; or the like.

According to some embodiments, the method may further comprise determining, by the computing system and using one of the at least one AI/ML model, a structure of the interaction with the user, based at least in part on the identified one or more goals of the conversation; and determining, by the computing system and using one of the at least one AI/ML model, one or more first parameters for the determined structure of the interaction with the user, the one or more first parameters defining conversational guardrails for steering the interaction away from conversational tangents and toward achieving the at least one goal of the conversation. In some cases, the one or more first conversational threads may be generated based on the one or more first parameters.

In some embodiments, the method may further comprise mapping, by the computing system and using one of the at least one AI/ML model, a flow of the interaction with the user; and based on a determination that the flow of the interaction is moving away from achieving the at least one goal of the conversation: determining, by the computing system and using one of the at least one AI/ML model, one or more second parameters for steering the interaction back toward achieving the at least one goal of the conversation; generating, by the computing system and using one of the at least one AI/ML model, one or more second conversational threads configured to steer the interaction back toward achieving the at least one goal of the conversation, based on the one or more second parameters; and causing, by the computing system, the at least one AI/ML-driven persona to continue the conversation with the user using the one or more second conversational threads.

According to some embodiments, the method may further comprise, during the interaction with the user, performing, by the computing system and using one of the at least one AI/ML model, at least one of continuous tone analysis or continuous sentiment analysis of the interaction with the user; and after the interaction with the user, updating, by the computing system, the at least one AI/ML model based on the at least one of continuous tone analysis or continuous sentiment analysis of the interaction with the user.

In another aspect, a system may comprise a computing system, which may comprise at least one first processor and a first non-transitory computer readable medium communicatively coupled to the at least one first processor. The first non-transitory computer readable medium may have stored thereon computer software comprising a first set of instructions that, when executed by the at least one first processor, causes the computing system to: cause at least one artificial intelligence (“AI”)/machine learning (“ML”)-driven persona to interact with a user via a user interface (“UI”), the interaction including a conversation between the at least one AI/ML-driven persona and the user; analyze, using one of at least one AI/ML model, the conversation to identify one or more goals of the conversation related to at least one of one or more products or one or more services provided by a provider; generate, using one of the at least one AI/ML model, one or more first conversational threads configured to achieve at least one goal of the conversation among the one or more goals of the conversation related to the at least one of the one or more products or the one or more services; and cause the at least one AI/ML-driven persona to continue the conversation with the user using the one or more first conversational threads to work toward achieving the at least one goal of the conversation.

In yet another aspect, a method may comprise causing, by a computing system, at least one artificial intelligence (“AI”)/machine learning (“ML”)-driven persona to interact with a user via a user interface (“UI”), the interaction including a conversation between the at least one AI/ML-driven persona and the user; analyzing, by the computing system and using one of at least one AI/ML model, the conversation to identify one or more goals of the conversation; determining, by the computing system and using one of the at least one AI/ML model, a structure of the interaction with the user, based at least in part on the identified one or more goals of the conversation; generating, by the computing system and using one of the at least one AI/ML model, one or more first conversational threads configured to achieve at least one goal of the conversation among the one or more goals of the conversation within the determined structure of the interaction with the user; causing, by the computing system, the at least one AI/ML-driven persona to continue the conversation with the user using the one or more first conversational threads to work toward achieving the at least one goal of the conversation; mapping, by the computing system and using one of the at least one AI/ML model, a flow of the interaction with the user; based on a determination that the flow of the interaction is moving away from achieving the at least one goal of the conversation, generating, by the computing system and using one of the at least one AI/ML model, one or more second conversational threads configured to steer the interaction back toward achieving the at least one goal of the conversation; and causing, by the computing system, the at least one AI/ML-driven persona to continue the conversation with the user using the one or more second conversational threads to steer the interaction back toward achieving the at least one goal of the conversation.

In some embodiments, the method may further comprise determining, by the computing system and using one of the at least one AI/ML model, one or more first parameters for the determined structure of the interaction with the user, the one or more first parameters defining conversational guardrails for steering the interaction away from conversational tangents and toward achieving the at least one goal of the conversation, wherein the one or more first conversational threads may be generated based on the one or more first parameters; and determining, by the computing system and using one of the at least one AI/ML model, one or more second parameters for steering the interaction back toward achieving the at least one goal of the conversation, wherein the one or more second conversational threads may be generated based on the one or more second parameters.

According to some embodiments, the method may further comprise analyzing, by the computing system and using one of the at least one AI/ML model, the interaction to identify one or more observable characteristics of the user, the one or more observable characteristics including at least one of one or more speech patterns of the user, a language used by the user, whether the user has an accent, what accent the user has, whether English is a second language for the user, one or more non-verbal cues of the user, a demeanor of the user, a sentiment of the user, or an emotional state of the user, and/or the like; accessing and analyzing, by the computing system and using one of the at least one AI/ML model, stored information associated with the user to identify one or more conversation points, the stored information including at least one of account information associated with the user, contact information associated with the user, previous interactions with the user, historical data associated with the user, demographic information about the user, personal information about the user, user-volunteered information regarding general interests of the user, information regarding a market segment within which the user is classified, or societal information for a societal segment to which the user belongs, and/or the like; and causing, by the computing system, the at least one AI/ML-driven persona to adapt by modifying the interaction with the user, based at least in part on at least one of the identified one or more observable characteristics of the user or the identified one or more conversation points, to enhance or improve the interaction with the user.

In an aspect, a method may comprise causing, by a computing system, at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning a skill; analyzing, by the computing system and using one of at least one AI/ML model, the interaction to determine a first skill level of the user in the skill, the first skill level comprising at least one of a level of knowledge of the skill that the user possesses, a level of understanding of the skill by the user, or a level of ability of the user to apply the skill under one or more conditions, and/or the like; generating, by the computing system and using one of the at least one AI/ML model, one or more first conversational threads configured to achieve a first goal of improving the first skill level of the user in the skill, based on the analysis of the interaction and based on instructional data for the skill that is accessible from a database; and causing, by the computing system, the at least one AI/ML-driven persona to continue the conversation with the user using the one or more first conversational threads to work toward achieving the first goal.

In some embodiments, the computing system may comprise at least one of a server, an AI system, a ML system, an AI/ML system, a deep learning (“DL”) system, a user interactive system, a customer interface server, a skill training system, an automated tutoring system, an education server, an education facility computing system, a cloud computing system, or a distributed computing system, and/or the like. In some cases, the UI may comprise one of a voice-only UI, a telephone communication UI, a video-only UI, a video with voice UI, a chat UI, a software application (“app”) UI, a holographic UI, a virtual reality (“VR”)-based UI, an augmented reality (“AR”)-based UI, a mixed reality (“MR”)-based UI, or a web-portal-based UI, or the like.

According to some embodiments, the skill may be among a plurality of skills comprising at least one of skill in a language among a plurality of languages, skill in understanding a culture among a plurality of cultures, skill in living in nature, skill in living in small population centers, skill in living in large population centers, skill in living in rural areas, skill in living in cities, skill in navigating tourist areas, skill in an elementary school course, skill in a middle school course, skill in a high school course, skill in a college course, skill in a university course, skill in a vocational course, skill in mathematics, skill in physics, skill in chemistry, skill in biology, skill in sociology, skill in philosophy, skill in psychology, skill in computer programming, skill in literary studies, skill in linguistics, skill in writing, skill in civics or social studies, skill in historical studies, skill in geography, skill in geology, skill in engineering, skill in educating others, skill in training animals, skill in home repairs, skill in vehicle repairs, skill in appliance repairs, skill in computer repairs, skill in mobile device repairs, skill in assembling a consumer product, skill in construction, skill in driving, skill in sports activity, skill in recreational activity, skill in card games, skill in board games, skill in trivia games, skill in cooking, skill in butchering meats, skill in cleaning, skill in crafting, skill in carpentry, skill in metalworking, skill in smithing, skill in artistic forms, skill in painting, skill in first aid, skill in navigation, skill in buying, skill in selling, skill in bartering, skill in negotiation, skill in trading, skill in marketing, skill in accounting, skill in business development, skill in public speaking, skill in giving presentations, skill in communication, skill in management, or skill in leadership, and/or the like.

In some embodiments, the method may further comprise at least one of: using a first set of AI/ML-driven personas to assist the user in learning a first skill among the plurality of skills; and using a second set of AI/ML-driven personas that is different from the first set of AI/ML-driven personas to assist the user in learning a second skill among the plurality of skills that is different from the first skill; or using a first set of learning strategies among a plurality of learning strategies for assisting the user in learning the first skill; and using a second set of learning strategies among the plurality of learning strategies that is different from the first set of learning strategies for assisting the user in learning the second skill, wherein the plurality of learning strategies includes rote learning, learning using flash cards or virtual flash cards, learning by interaction, learning by practice, auditory learning, visual learning, or learning using a combination of two or more of said learning strategies; and/or the like.

According to some embodiments, assisting the user in learning the skill may be based on a lesson plan that is generated based on the instructional data for the skill. In some examples, the method may further comprise updating, by the computing system and using one of the at least one AI/ML model, the lesson plan to generate an updated lesson plan, based on the analysis of the interaction and based on instructional data for the skill, wherein the one or more first conversational threads are further based on the updated lesson plan; analyzing, by the computing system and using one of the at least one AI/ML model, the continued conversation to determine effectiveness of the updated lesson plan, by determining whether there are any changes to the first skill level of the user in the skill, after use of the one or more first conversational threads; adapting, by the computing system and using one of the at least one AI/ML model, the updated lesson plan to generate an adapted lesson plan, based on the analysis of the continued conversation and based on the instructional data for the skill; generating, by the computing system and using one of the at least one AI/ML model, one or more second conversational threads based on the adapted lesson plan; and causing, by the computing system, the at least one AI/ML-driven persona to continue the conversation with the user using the one or more second conversational threads.

In some instances, the skill may be skill in a first language among a plurality of languages, wherein analyzing the conversation or the continued conversation may comprise at least one of: analyzing, by the computing system and using one of at least one AI/ML model, the interaction to determine at least one of a breadth of a breadth of listening vocabulary, a depth of listening vocabulary, a breadth of speaking vocabulary, a depth of speaking vocabulary, reading vocabulary, a depth of reading vocabulary, a breadth of writing vocabulary, or a depth of writing vocabulary that the user has in the first language, and/or the like; analyzing, by the computing system and using one of at least one AI/ML model, the interaction to determine the user's proficiency and understanding of grammar in the first language, the grammar including at least one of tense, number, noun classes, locative relations, syntax, grammatical structure, or grammatical gender, and/or the like; analyzing, by the computing system and using one of at least one AI/ML model, the interaction to determine the user's pronunciation of words in the first language; analyzing, by the computing system and using one of at least one AI/ML model, the interaction to determine an optimal learning strategy among a plurality of learning strategies for the user to learn the first language, the plurality of learning strategies including learning by flash cards or virtual flash cards, learning by usage in sentences, learning by vocabulary drills, learning by exposure to particular words in different contexts, learning by language immersion, learning by interaction, learning by reading sentences or passages, learning by speaking sentences, learning by listening to conversations, auditory learning, visual learning, learning by translating to a second language among the plurality of languages that is different from the first language, or learning using a combination of two or more of said learning strategies, and/or the like; analyzing, by the computing system and using one of at least one AI/ML model, the interaction to determine how quickly the user interacts or responds; or analyzing, by the computing system and using one of at least one AI/ML model, the interaction to determine which AI/ML-driven persona or which combination of two or more AI/ML-driven personas best assists the user in learning the first language; and/or the like.

In some cases, updating the lesson plan or adapting the updated lesson plan may comprise at least one of: changing, by the computing system, one or more first AI/ML-driven personas among the at least one AI/ML-driven persona to one or more second AI/ML-driven persona among the at least one AI/ML-driven persona; changing, by the computing system, at least one of a voice or speech pattern, a tone, an accent, a pitch, a cadence, or a gender of the at least one AI/ML-driven persona, and/or the like; changing, by the computing system, at least one of a time of day, a time of week, or a time of month that the lesson plan or the updated lesson plan is implemented, and/or the like; changing, by the computing system, a pace of the lesson plan or the updated lesson plan; or changing, by the computing system, a learning strategy among a plurality of learning strategies from a first learning strategy to a second learning strategy, the plurality of learning strategies including rote learning, learning using flash cards or virtual flash cards, learning by interaction, learning by practice, auditory learning, visual learning, or learning using a combination of two or more of said learning strategies, and/or the like; and/or the like.

In some embodiments, the method may further comprise analyzing, by the computing system and using one of the at least one AI/ML model, the interaction to identify one or more observable characteristics of the user, the one or more observable characteristics including at least one of one or more speech patterns of the user, a language used by the user, whether the user has an accent, what accent the user has, one or more non-verbal cues of the user, a demeanor of the user, a sentiment of the user, or an emotional state of the user, and/or the like; accessing and analyzing, by the computing system and using one of the at least one AI/ML model, stored information associated with the user to identify one or more conversation points, the stored information including at least one of account information associated with the user, contact information associated with the user, order history data associated with the user, previous interactions with the user, historical data associated with the user, demographic information about the user, personal information about the user, user-volunteered information regarding general interests of the user, information regarding a market segment within which the user is classified, or societal information for a societal segment to which the user belongs, and/or the like; and causing, by the computing system, the at least one AI/ML-driven persona to adapt by modifying the interaction with the user, based at least in part on at least one of the identified one or more observable characteristics of the user or the identified one or more conversation points, to enhance or improve the interaction with the user.

According to some embodiments, the at least one AI/ML-driven persona is among a plurality of AI/ML-driven personas comprising at least one of one or more personas based on a fictional literary character, one or more personas based on a non-fictional literary character, one or more personas based on a comic-book character, one or more personas based on a cartoon character, one or more personas based on an anime character, one or more personas based on a manga character, one or more personas based on a television character, one or more personas based on a movie character, one or more personas based on a character from an advertisement, one or more personas based on a mascot, one or more personas based on a meme, one or more personas based on an athlete, one or more personas based on a sports personality, one or more personas based on a news personality, one or more personas based on a political personality, one or more personas based on a reality television personality, one or more personas based on a social media influencer, one or more personas based on a living celebrity, one or more personas based on a deceased celebrity, one or more personas based on a historical figure, one or more personas based on a fictionalization of a historical figure, one or more personas based on a character played by an actor or actress, one or more personas based on a bespoke character, or one or more personas simulating average humans in a geographical area within which the user is currently located or was previously residing, and/or the like.

In some cases, the UI may be a visual-based UI, wherein the method may further comprise generating, by the computing system, an avatar for each of the at least one AI/ML-driven persona; displaying, by the computing system and within the UI, the avatar for each of the at least one AI/ML-driven persona; and animating, by the computing system and within the UI, the avatar in synchronization with the conversation with the user, wherein the interaction may further comprise the animation of the avatar.

In some instances, each AI/ML-driven persona may have a set personality, the set personality including at least one of a set speech pattern, a set mannerism, a set command of one or more languages, a set accent, a set collection of non-verbal cues, or a set collection of emotional demeanors, and/or the like. In some examples, each of the interaction, the conversation, the one or more first conversational threads, and the animation of each AI/ML-driven persona may be performed in a manner consistent with the set personality of said AI/ML-driven persona.

In some examples, the method may further comprise, in response to user selection of a gamification mode, performing the following: generating, by the computing system, a visual representation of a list of one or more goals for learning the skill, the one or more goals for learning the skill including the first goal; and in response to achieving a goal among the one or more goals for learning the skill, generating, by the computing system, an animation of one or more of the at least one AI/ML-driven persona performing one or more actions comprising checking off the achieved goal, removing the achieved goal from the list, or marking the achieved goal as having been achieved, the one or more actions being performed in a manner consistent with the set personality of each of the one or more of the at least one AI/ML-driven persona.

In some instances, the method may further comprise, prior to the conversation or during a setup phase, performing one of: receiving, by the computing system, a user selection of the at least one AI/ML-driven persona from a licensed set of AI/ML-driven personas among the plurality of AI/ML-driven personas with whom to interact; identifying, by the computing system, the user, and selecting, by the computing system, the at least one AI/ML-driven persona from the licensed set of AI/ML-driven personas to match the user for interacting with the user, based on information regarding the identified user; setting, by the computing system, a default set of AI/ML-driven personas from the licensed set of AI/ML-driven personas, wherein the default set of AI/ML-driven personas comprises the at least one AI/ML-driven persona; or randomly selecting, by the computing system, the at least one AI/ML-driven persona from the licensed set of AI/ML-driven personas for interacting with the user; or the like.

In some cases, the method may further comprise at least one of: adapting or adjusting, by the computing system and using one of the at least one AI/ML model, a personality of one or more of the at least one AI/ML-driven persona to mold to or match a determined personality of the user, wherein the personality of the user may be determined based on at least one of analysis of the interaction with the user, analysis of a previous interaction with the user, or known information about the user; or adapting or adjusting, by the computing system and using one of the at least one AI/ML model, one or more interaction characteristics of one or more of the at least one AI/ML-driven persona to match to a determined corresponding interaction characteristic of the user, the one or more interaction characteristics including at least one of speech pattern, language, accent, cultural mannerisms, cultural phraseology, general mannerisms, general phraseology, slang, jargon, or sentiment, and/or the like; or the like.

In some embodiments, the method may further comprise determining, by the computing system and using one of the at least one AI/ML model, a structure of the interaction with the user, based at least in part on the first goal; and determining, by the computing system and using one of the at least one AI/ML model, one or more first parameters for the determined structure of the interaction with the user, the one or more first parameters defining conversational guardrails for steering the interaction away from conversational tangents and toward achieving the first goal. In some cases, the one or more first conversational threads may be generated based on the one or more first parameters.

According to some embodiments, the method may further comprise mapping, by the computing system and using one of the at least one AI/ML model, a flow of the interaction with the user; and based on a determination that the flow of the interaction is moving away from achieving the first goal: determining, by the computing system and using one of the at least one AI/ML model, one or more third parameters for steering the interaction back toward achieving the first goal; generating, by the computing system and using one of the at least one AI/ML model, one or more third conversational threads configured to steer the interaction back toward achieving the first goal, based on the one or more third parameters; and causing, by the computing system, the at least one AI/ML-driven persona to continue the conversation with the user using the one or more third conversational threads.

In another aspect, a system may comprise a computing system, which may comprise at least one first processor and a first non-transitory computer readable medium communicatively coupled to the at least one first processor. The first non-transitory computer readable medium may have stored thereon computer software comprising a first set of instructions that, when executed by the at least one first processor, causes the computing system to: cause at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning a skill; analyze, using one of at least one AI/ML model, the interaction to determine a first skill level of the user in the skill, the first skill level comprising at least one of a level of knowledge of the skill that the user possesses, a level of understanding of the skill by the user, or a level of ability of the user to apply the skill under one or more conditions, and/or the like; generate, using one of the at least one AI/ML model, one or more first conversational threads configured to achieve a first goal of improving the first skill level of the user in the skill, based on the analysis of the interaction and based on instructional data for the skill that is accessible from a database; and cause the at least one AI/ML-driven persona to continue the conversation with the user using the one or more second conversational threads to work toward achieving the first goal.

In yet another aspect, a method may comprise causing, by a computing system, at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning a first language among a plurality of languages; analyzing, by the computing system and using one of at least one AI/ML model, the interaction to determine a first skill level of the user in the first language, the first skill level comprising at least one of a level of knowledge of the first language that the user possesses, a level of understanding of the first language by the user, or a level of ability of the user to apply the first language under one or more conditions, and/or the like; generating, by the computing system and using one of the at least one AI/ML model, one or more first conversational threads configured to achieve a first goal of improving the first skill level of the user in the first language, based on the analysis of the interaction and based on instructional data for the first language that is accessible from a database; and causing, by the computing system, the at least one AI/ML-driven persona to continue the conversation with the user using the one or more first conversational threads to work toward achieving the first goal.

In some embodiments, assisting the user in learning the first language may be based on a lesson plan that is generated based on the instructional data for the first language, wherein the method may further comprise updating, by the computing system and using one of the at least one AI/ML model, the lesson plan to generate an updated lesson plan, based on the analysis of the interaction and based on instructional data for the first language, wherein the one or more first conversational threads are further based on the updated lesson plan; analyzing, by the computing system and using one of the at least one AI/ML model, the continued conversation to determine effectiveness of the updated lesson plan, by determining whether there are any changes to the first skill level of the user in the first language the first language the first language the first language, after use of the one or more first conversational threads; adapting, by the computing system and using one of the at least one AI/ML model, the updated lesson plan to generate an adapted lesson plan, based on the analysis of the continued conversation and based on the instructional data for the first language; generating, by the computing system and using one of the at least one AI/ML model, one or more second conversational threads based on the adapted lesson plan; and causing, by the computing system, the at least one AI/ML-driven persona to continue the conversation with the user using the one or more second conversational threads.

According to some embodiments, analyzing the conversation or the continued conversation may comprise at least one of: analyzing, by the computing system and using one of at least one AI/ML model, the interaction to determine at least one of a breadth of a breadth of listening vocabulary, a depth of listening vocabulary, a breadth of speaking vocabulary, a depth of speaking vocabulary, reading vocabulary, a depth of reading vocabulary, a breadth of writing vocabulary, or a depth of writing vocabulary that the user has in the first language, and/or the like; analyzing, by the computing system and using one of at least one AI/ML model, the interaction to determine the user's proficiency and understanding of grammar in the first language, the grammar including at least one of tense, number, noun classes, locative relations, syntax, grammatical structure, or grammatical gender, and/or the like; analyzing, by the computing system and using one of at least one AI/ML model, the interaction to determine the user's pronunciation of words in the first language; analyzing, by the computing system and using one of at least one AI/ML model, the interaction to determine an optimal learning strategy among a plurality of learning strategies for the user to learn the first language, the plurality of learning strategies including learning by flash cards or virtual flash cards, learning by usage in sentences, learning by vocabulary drills, learning by exposure to particular words in different contexts, learning by language immersion, learning by interaction, learning by reading sentences or passages, learning by speaking sentences, learning by listening to conversations, auditory learning, visual learning, learning by translating to a second language among the plurality of languages that is different from the first language, or learning using a combination of two or more of said learning strategies, and/or the like; analyzing, by the computing system and using one of at least one AI/ML model, the interaction to determine how quickly the user interacts or responds; or analyzing, by the computing system and using one of at least one AI/ML model, the interaction to determine which AI/ML-driven persona or which combination of two or more AI/ML-driven personas best assists the user in learning the first language; and/or the like.

In an aspect, a method may comprise causing, by a computing system, at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning one or more social skills to interact with other people; analyzing, by the computing system and using one of at least one AI/ML model, the interaction to determine a first skill level of the user in at least one set of social skills among the one or more social skills, the first skill level comprising a level of competence in the at least one set of social skill that the user possesses; generating, by the computing system and using one of the at least one AI/ML model, one or more first conversational threads configured to achieve a first goal of developing the first skill level of the user in the at least one set of social skill, based on the analysis of the interaction and based on social or behavioral therapy data for the at least one set of social skills that is accessible from a database; and causing, by the computing system, the at least one AI/ML-driven persona to continue the conversation with the user using the one or more first conversational threads to work toward achieving the first goal.

In some embodiments, the computing system may comprise at least one of a server, an AI system, a ML system, an AI/ML system, a deep learning (“DL”) system, a user interactive system, a customer interface server, a social skills training system, a behavioral skills training system, a behavioral therapy system, an education server, an education facility computing system, a cloud computing system, or a distributed computing system, and/or the like. In some cases, the UI may comprise one of a voice-only UI, a telephone communication UI, a video-only UI, a video with voice UI, a chat UI, a software application (“app”) UI, a holographic UI, a virtual reality (“VR”)-based UI, an augmented reality (“AR”)-based UI, a mixed reality (“MR”)-based UI, or a web-portal-based UI, and/or the like.

According to some embodiments, the one or more social skills may comprise at least one of social coordination skills, mentoring skills, negotiation skills, persuasion skills, psychosocial service orientation skills, social perceptiveness skills, active listening skills, delegation skills, decision-making skills, problem-solving skills, creative thinking skills, critical thinking skills, communication skills, interpersonal skills, self-awareness skills, empathy skills, assertiveness skills, equanimity skills, psychological resilience skills, or coping skills, and/or the like.

In some embodiments, the method may further comprise at least one of: using a first set of AI/ML-driven personas to assist the user in learning a first set of social skills among the one or more social skills; and using a second set of AI/ML-driven personas that is different from the first set of AI/ML-driven personas to assist the user in learning a second set of social skill among the one or more social skills that is different from the first set of social skills; or using a first set of social training strategies among a plurality of social training strategies for assisting the user in learning the first set of social skills; and using a second set of social training strategies among the plurality of social training strategies that is different from the first set of social training strategies for assisting the user in learning the second set of social skills, wherein the plurality of social training strategies includes encouragement of use of at least one of self-reflection, meditation, social setting simulation, empathetic interaction, behavioral adjustment, conversation training, psychotherapy treatment, cognitive behavioral therapy (“CBT”) or computerized CBT, dialectical behavior therapy, simulated hypnotherapy, art therapy, or learning using a combination of two or more of said social training strategies, and/or the like.

According to some embodiments, assisting the user in learning the one or more social skills may be based on a therapy plan that is generated based on the social or behavioral therapy data for the at least one set of social skills, wherein the method may further comprise updating, by the computing system and using one of the at least one AI/ML model, the therapy plan to generate an updated therapy plan, based on the analysis of the interaction and based on social or behavioral therapy data for the at least one set of social skills, wherein the one or more first conversational threads are further based on the updated therapy plan; analyzing, by the computing system and using one of the at least one AI/ML model, the continued conversation to determine effectiveness of the updated therapy plan, by determining whether there are any changes to the first skill level of the user in the at least one set of social skills, after use of the one or more first conversational threads; adapting, by the computing system and using one of the at least one AI/ML model, the updated therapy plan to generate an adapted therapy plan, based on the analysis of the continued conversation and based on the social or behavioral therapy data for the at least one set of social skills; generating, by the computing system and using one of the at least one AI/ML model, one or more second conversational threads based on the adapted therapy plan; and causing, by the computing system, the at least one AI/ML-driven persona to continue the conversation with the user using the one or more second conversational threads.

In some embodiments, the at least one set of social skills may be a skill for interacting with multiple people at a time, wherein analyzing the conversation or the continued conversation may comprise at least one of: analyzing, by the computing system and using one of at least one AI/ML model, the interaction to determine a level of social anxiety that the user possesses; or analyzing, by the computing system and using one of at least one AI/ML model, the interaction to identify one or more triggers for the user's discomfort arising from social anxiety. In some instances, the method may further comprise at least one of: causing, by the computing system, a plurality of different AI/ML-driven personas to simulate a crowd of people whose number is determined to be one of the identified one or more triggers; implementing, by the computing system, a therapy plan in which the user is introduced to a different sets of AI/ML-driven personas, each successive set increasing in number of different AI/ML-driven personas as the user becomes more comfortable interacting with each set of AI/ML-driven personas, to assist the user in becoming desensitized to crowd triggers for social anxiety; or simulating, by the computing system, a social setting in which the user is planning to speak, by filling the simulated social setting with a plurality of different AI/ML-driven personas that are caused to interact with the user within the context of the social setting.

According to some embodiments, updating the therapy plan or adapting the updated therapy plan may comprise at least one of: changing, by the computing system, one or more first AI/ML-driven personas among the at least one AI/ML-driven persona to one or more second AI/ML-driven persona among the at least one AI/ML-driven persona; changing, by the computing system, at least one of a voice or speech pattern, a tone, an accent, a pitch, a cadence, or a gender of the at least one AI/ML-driven persona, and/or the like; changing, by the computing system, at least one of a time of day, a time of week, or a time of month, and/or the like, that the therapy plan or the updated therapy plan is implemented; changing, by the computing system, a pace of the therapy plan or the updated therapy plan; or changing, by the computing system, a set of social training strategies among a plurality of social training strategies from a first set of social training strategies to a second set of social training strategies, the plurality of social training strategies including encouragement of use of at least one of self-reflection, meditation, social setting simulation, empathetic interaction, behavioral adjustment, conversation training, psychotherapy treatment, cognitive behavioral therapy (“CBT”) or computerized CBT, dialectical behavior therapy, simulated hypnotherapy, art therapy, or learning using a combination of two or more of said social training strategies, and/or the like.

In some embodiments, the method may further comprise analyzing, by the computing system and using one of the at least one AI/ML model, the interaction to identify one or more observable characteristics of the user, the one or more observable characteristics including at least one of one or more speech patterns of the user, a language used by the user, whether the user has an accent, what accent the user has, one or more non-verbal cues of the user, a demeanor of the user, a sentiment of the user, or an emotional state of the user, and/or the like; accessing and analyzing, by the computing system and using one of the at least one AI/ML model, stored information associated with the user to identify one or more conversation points, the stored information including at least one of account information associated with the user, contact information associated with the user, order history data associated with the user, previous interactions with the user, historical data associated with the user, demographic information about the user, personal information about the user, user-volunteered information regarding general interests of the user, information regarding a market segment within which the user is classified, or societal information for a societal segment to which the user belongs, and/or the like; and causing, by the computing system, the at least one AI/ML-driven persona to adapt by modifying the interaction with the user, based at least in part on at least one of the identified one or more observable characteristics of the user or the identified one or more conversation points, to enhance or improve the interaction with the user.

According to some embodiments, the at least one AI/ML-driven persona may be among a plurality of AI/ML-driven personas comprising at least one of one or more personas based on a fictional literary character, one or more personas based on a non-fictional literary character, one or more personas based on a comic-book character, one or more personas based on a cartoon character, one or more personas based on an anime character, one or more personas based on a manga character, one or more personas based on a television character, one or more personas based on a movie character, one or more personas based on a character from an advertisement, one or more personas based on a mascot, one or more personas based on a meme, one or more personas based on an athlete, one or more personas based on a sports personality, one or more personas based on a news personality, one or more personas based on a political personality, one or more personas based on a reality television personality, one or more personas based on a social media influencer, one or more personas based on a living celebrity, one or more personas based on a deceased celebrity, one or more personas based on a historical figure, one or more personas based on a fictionalization of a historical figure, one or more personas based on a character played by an actor or actress, one or more personas based on a bespoke character, or one or more personas simulating average humans in a geographical area within which the user is currently located or was previously residing, and/or the like.

In some embodiments, the UI may be a visual-based UI, wherein the method may further comprise generating, by the computing system, an avatar for each of the at least one AI/ML-driven persona; displaying, by the computing system and within the UI, the avatar for each of the at least one AI/ML-driven persona; and animating, by the computing system and within the UI, the avatar in synchronization with the conversation with the user, wherein the interaction may further comprise the animation of the avatar.

According to some embodiments, each AI/ML-driven persona may have a set personality, the set personality including at least one of a set speech pattern, a set mannerism, a set command of one or more languages, a set accent, a set collection of non-verbal cues, or a set collection of emotional demeanors, and/or the like. In some cases, each of the interaction, the conversation, the one or more first conversational threads, and the animation of each AI/ML-driven persona may be performed in a manner consistent with the set personality of said AI/ML-driven persona.

In some embodiments, the method may further comprise, in response to user selection of a gamification mode, performing the following: generating, by the computing system, a visual representation of a list of one or more goals for learning the at least one set of social skills, the one or more goals for learning the at least one set of social skills including the first goal; and in response to achieving a goal among the one or more goals for learning the at least one set of social skills, generating, by the computing system, an animation of one or more of the at least one AI/ML-driven persona performing one or more actions comprising checking off the achieved goal, removing the achieved goal from the list, or marking the achieved goal as having been achieved, the one or more actions being performed in a manner consistent with the set personality of each of the one or more of the at least one AI/ML-driven persona.

According to some embodiments, the method may further comprise, prior to the conversation or during a setup phase, performing one of: receiving, by the computing system, a user selection of the at least one AI/ML-driven persona from a licensed set of AI/ML-driven personas among the plurality of AI/ML-driven personas with whom to interact; identifying, by the computing system, the user, and selecting, by the computing system, the at least one AI/ML-driven persona from the licensed set of AI/ML-driven personas to match the user for interacting with the user, based on information regarding the identified user; identifying, by the computing system, the user, and selecting, by the computing system, the at least one AI/ML-driven persona from the licensed set of AI/ML-driven personas to match a personality of a person whom the user is determined to respect or feel comfortable talking with for interacting with the user, based on information regarding the identified user; setting, by the computing system, a default set of AI/ML-driven personas from the licensed set of AI/ML-driven personas, wherein the default set of AI/ML-driven personas comprises the at least one AI/ML-driven persona; or randomly selecting, by the computing system, the at least one AI/ML-driven persona from the licensed set of AI/ML-driven personas for interacting with the user; or the like.

In some embodiments, the method may further comprise at least one of: adapting or adjusting, by the computing system and using one of the at least one AI/ML model, a personality of one or more of the at least one AI/ML-driven persona to mold to or match a determined personality of the user, wherein the personality of the user may be determined based on at least one of analysis of the interaction with the user, analysis of a previous interaction with the user, or known information about the user, and/or the like; adapting or adjusting, by the computing system and using one of the at least one AI/ML model, one or more interaction characteristics of one or more of the at least one AI/ML-driven persona to match to a determined corresponding interaction characteristic of the user, the one or more interaction characteristics including at least one of speech pattern, language, accent, cultural mannerisms, cultural phraseology, general mannerisms, general phraseology, slang, jargon, or sentiment, and/or the like; adapting or adjusting, by the computing system and using one of the at least one AI/ML model, a personality of one or more of the at least one AI/ML-driven persona to mold to or match a personality of a person whom the user is determined to respect or feel comfortable talking with, wherein the personality of the person whom the user is determined to respect or feel comfortable talking with is determined based on at least one of analysis of the interaction with the user, analysis of a previous interaction with the user, or known information about the user, and/or the like; or adapting or adjusting, by the computing system and using one of the at least one AI/ML model, one or more interaction characteristics of one or more of the at least one AI/ML-driven persona to match to a determined corresponding interaction characteristic of the person whom the user is determined to respect or feel comfortable talking with, the one or more interaction characteristics including at least one of speech pattern, language, accent, cultural mannerisms, cultural phraseology, general mannerisms, general phraseology, slang, jargon, or sentiment, and/or the like; and/or the like.

According to some embodiments, the method may further comprise determining, by the computing system and using one of the at least one AI/ML model, a structure of the interaction with the user, based at least in part on the first goal; and determining, by the computing system and using one of the at least one AI/ML model, one or more first parameters for the determined structure of the interaction with the user, the one or more first parameters defining conversational guardrails for steering the interaction away from conversational tangents and toward achieving the first goal. In some instances, the one or more first conversational threads may be generated based on the one or more first parameters.

In some embodiments, the method may further comprise mapping, by the computing system and using one of the at least one AI/ML model, a flow of the interaction with the user; and based on a determination that the flow of the interaction is moving away from achieving the first goal: determining, by the computing system and using one of the at least one AI/ML model, one or more third parameters for steering the interaction back toward achieving the first goal; generating, by the computing system and using one of the at least one AI/ML model, one or more third conversational threads configured to steer the interaction back toward achieving the first goal, based on the one or more third parameters; and causing, by the computing system, the at least one AI/ML-driven persona to continue the conversation with the user using the one or more third conversational threads.

In another aspect, a system may comprise a computing system, which may comprise at least one first processor and a first non-transitory computer readable medium communicatively coupled to the at least one first processor. The first non-transitory computer readable medium may have stored thereon computer software comprising a first set of instructions that, when executed by the at least one first processor, causes the computing system to: cause at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning one or more social skills to interact with other people; analyze, using one of at least one AI/ML model, the interaction to determine a first skill level of the user in at least one set of social skills among the one or more social skills, the first skill level comprising a level of competence in the at least one set of social skill that the user possesses; generate, using one of the at least one AI/ML model, one or more first conversational threads configured to achieve a first goal of developing the first skill level of the user in the at least one set of social skill, based on the analysis of the interaction and based on social or behavioral therapy data for the at least one set of social skills that is accessible from a database; and cause the at least one AI/ML-driven persona to continue the conversation with the user using the one or more first conversational threads to work toward achieving the first goal.

In yet another aspect, a method may comprise causing, by a computing system, at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning one or more social skills to interact with multiple people at a time; analyzing, by the computing system and using one of at least one AI/ML model, the interaction to determine a first skill level of the user in the one or more social skills, the first skill level comprising a level of competence in the one or more social skills that the user possesses; generating, by the computing system and using one of the at least one AI/ML model, one or more first conversational threads configured to achieve a first goal of developing the first skill level of the user in the one or more social skills, based on the analysis of the interaction and based on social or behavioral therapy data for the one or more social skills that is accessible from a database; and causing, by the computing system, the at least one AI/ML-driven persona to continue the conversation with the user using the one or more first conversational threads to work toward achieving the first goal.

In some embodiments, assisting the user in learning the one or more social skills to interact with multiple people at a time may be based on a therapy plan that is generated based on the social or behavioral therapy data for the one or more social skills, wherein the method may further comprise updating, by the computing system and using one of the at least one AI/ML model, the therapy plan to generate an updated therapy plan, based on the analysis of the interaction and based on social or behavioral therapy data for the one or more social skills, wherein the one or more first conversational threads may be further based on the updated therapy plan; analyzing, by the computing system and using one of the at least one AI/ML model, the continued conversation to determine effectiveness of the updated therapy plan, by determining whether there are any changes to the first skill level of the user in the one or more social skills, after use of the one or more first conversational threads; adapting, by the computing system and using one of the at least one AI/ML model, the updated therapy plan to generate an adapted therapy plan, based on the analysis of the continued conversation and based on the social or behavioral therapy data for the one or more social skills; generating, by the computing system and using one of the at least one AI/ML model, one or more second conversational threads based on the adapted therapy plan; and causing, by the computing system, the at least one AI/ML-driven persona to continue the conversation with the user using the one or more second conversational threads.

According to some embodiments, analyzing the conversation or the continued conversation may comprise at least one of: analyzing, by the computing system and using one of at least one AI/ML model, the interaction to determine a level of social anxiety that the user possesses; or analyzing, by the computing system and using one of at least one AI/ML model, the interaction to identify one or more triggers for the user's discomfort arising from social anxiety. In some examples, the method may further comprise at least one of: causing, by the computing system, a plurality of different AI/ML-driven personas to simulate a crowd of people whose number is determined to be one of the identified one or more triggers; implementing, by the computing system, a therapy plan in which the user is introduced to a different sets of AI/ML-driven personas, each successive set increasing in number of different AI/ML-driven personas as the user becomes more comfortable interacting with each set of AI/ML-driven personas, to assist the user in becoming desensitized to crowd triggers for social anxiety; or simulating, by the computing system, a social setting in which the user is planning to speak, by filling the simulated social setting with a plurality of different AI/ML-driven personas that are caused to interact with the user within the context of the social setting.

Various modifications and additions can be made to the embodiments discussed without departing from the scope of the invention. For example, while the embodiments described above refer to particular features, the scope of this invention also includes embodiments having different combination of features and embodiments that do not include all of the above-described features.

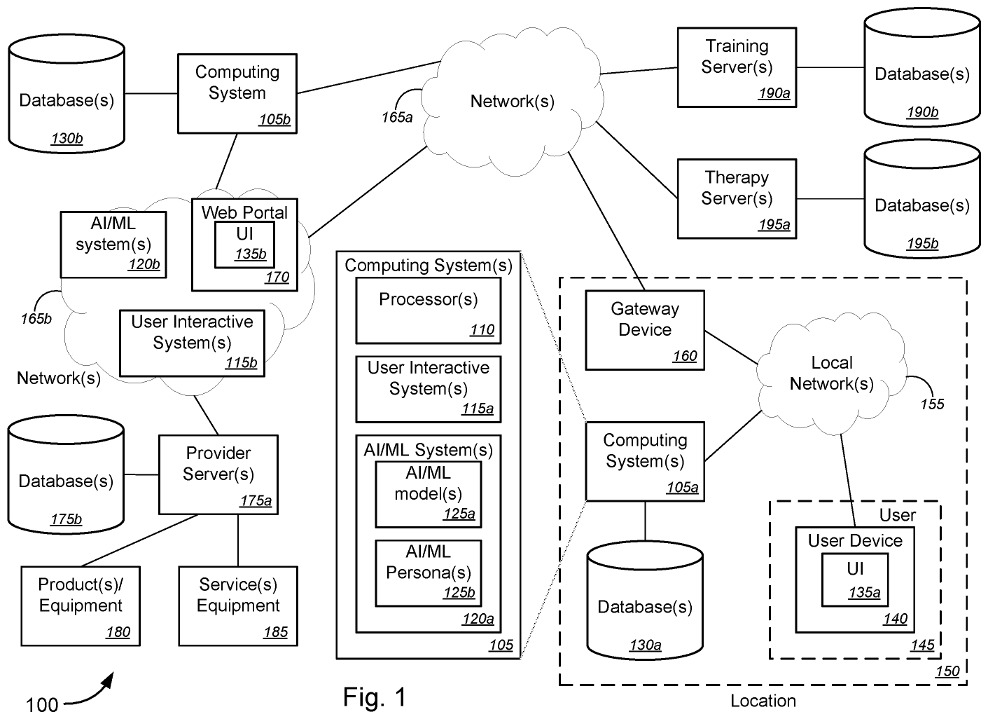

We now turn to the embodiments as illustrated by the drawings. FIGS. 1-6 illustrate some of the features of the method, system, and apparatus for implementing computerized human interaction systems, and, more particularly, to methods, systems, and apparatuses for implementing tailored synthetic personas with parameterized behaviors, as referred to above. The methods, systems, and apparatuses illustrated by FIGS. 1-6 refer to examples of different embodiments that include various components and steps, which can be considered alternatives or which can be used in conjunction with one another in the various embodiments. The description of the illustrated methods, systems, and apparatuses shown in FIGS. 1-6 is provided for purposes of illustration and should not be considered to limit the scope of the different embodiments.

### Specific Exemplary Embodiments

With reference to the figures, FIG. 1 is a schematic diagram illustrating a system 100 for implementing tailored synthetic personas with parameterized behaviors, in accordance with various embodiments.

In the non-limiting embodiment of FIG. 1, system 100 may include a computing system(s) 105, which may include computing system(s) 105a and/or computing system(s) 105b. In some cases, computing system(s) 105a and/or 105b may each include, without limitation, at least one of a processor(s) 110, a user interactive system(s) 115, and/or an artificial intelligence (“AI”)/machine learning (“ML”) system(s) 120, and/or the like. Each AI/ML system(s) 120 may include, but is not limited to, one or more AI/ML models 125a and one or more AI/ML-driven personas 125b. System 100 may further include a database(s) 130 (including database(s) 130a communicatively coupled to computing system(s) 105a and/or database(s) 130b communicatively coupled to computing system(s) 105b), a user interface (“UI”) 135a for a user device 140 that may be associated with a user 145, and the like. In some instances, the computing system(s) 105a, database(s) 130a, and user device 140 may be located at location 150, at which a local network(s) 155 may be established that communicates, via gateway device 160, to external network(s) 165a and/or 165b, or the like.

In some examples, user interactive system(s) 115 may include user interactive system(s) 115a that is integrated with, disposed within, or otherwise part of computing system(s) 105a and/or 105b, computing system(s) 105b being located within or communicatively coupled with one or more of network(s) 165a or 165b, or the like. Alternatively, or additionally, user interactive system(s) 115 may include user interactive system(s) 115b that is external to computing system(s) 105a and 105b, user interactive system(s) 115b being located within or communicatively coupled with one or more of network(s) 165a or 165b, or the like. Similarly, in some examples, AI/ML system(s) 120 may include AI/ML system(s) 120a that is integrated with, disposed within, or otherwise part of computing system(s) 105a and/or 105b, or the like. Alternatively, or additionally, AI/ML system(s) 120 may include AI/ML system(s) 120b that is external to computing system(s) 105a and 105b, AI/ML system(s) 120b being located within or communicatively coupled with one or more of network(s) 165a or 165b, or the like. System 100 may further include UI 135b for a web portal 170 that may run on a server (not shown) in network(s) 165b, or the like. The user interactive system(s) 115 controls, manages, and/or facilitates interaction between the computing system(s) 105 and the user, in some cases, by controlling, managing, and/or triggering the AI/ML system(s) 120 to interact with the user using one or more AI/ML personas 125b whose personalities and interactions may be refined based on training of the corresponding one or more AI/ML models 125a. In some cases, the AI/ML system(s) may be based on one or more of ML model(s), deep learning (“DL”) model(s), transformer ML/DL model(s), large language model(s) (“LLM(s)”), convolutional neural network (“CNN”) model(s), artificial neural network (“ANN”) model(s), recurrent neural network (“RNN”) model(s), and/or the like.

In some embodiments, system 100 may further include provider server(s) 175a and corresponding database(s) 175b, the provider server(s) 175a (i) providing a user (e.g., user 145, or the like) with access to, (ii) managing, and/or (iii) controlling one or more products or equipment 180 and/or one or more service(s) equipment 185, or the like. Alternatively, or additionally, system 100 may further include training server(s) 190a and corresponding database(s) 190b, the training server(s) 190a (i) curating, (ii) managing, or (iii) otherwise providing access to instructional data for one or more skills that may be stored in database(s) 190b, or the like. Alternatively, or additionally, system 100 may further include therapy server(s) 195a and corresponding database(s) 195b, the therapy server(s) 195a (i) curating, (ii) managing, or (iii) otherwise providing access to social or behavioral therapy data for one or more social skills that may be stored in database(s) 195b, or the like.

According to some embodiments, the computing system(s) 105, 105a, and/or 105b (where applicable) may each include, without limitation, at least one of a server, an AI system, a ML system, an AI/ML system (e.g., AI/ML system(s) 120, or the like), a deep learning (“DL”) system, a user interactive system (e.g., user interactive system(s) 115, or the like), a customer interface server, a skill training system, an automated tutoring system, an education server, an education facility computing system, a social skills training system, a behavioral skills training system, a behavioral therapy system, a cloud computing system, or a distributed computing system, and/or the like. In some cases, the UI 135a and/or 135b may each include, but is not limited to, one of a voice-only UI, a telephone communication UI, a video-only UI, a video with voice UI, a chat UI, a software application (“app”) UI, a holographic UI, a virtual reality (“VR”)-based UI, an augmented reality (“AR”)-based UI, a mixed reality (“MR”)-based UI, or a web-portal-based UI, and/or the like. In some instances, unless otherwise specified, the UI 135a and/or 135b may each further include an audio UI that may be used in conjunction with each of the chat UI, the app UI, the holographic UI, the VR-based UI, the AR-based UI, the MR-based UI, and/or the web-portal-based UI, and/or the like.

In some embodiments, the one or more AI/ML-driven personas 125b may be among a plurality of AI/ML-driven personas including, but not limited to, at least one of one or more personas based on a fictional literary character, one or more personas based on a non-fictional literary character, one or more personas based on a comic-book character, one or more personas based on a cartoon character, one or more personas based on an anime character, one or more personas based on a manga character, one or more personas based on a television character, one or more personas based on a movie character, one or more personas based on a character from an advertisement, one or more personas based on a mascot, one or more personas based on a meme, one or more personas based on an athlete, one or more personas based on a sports personality, one or more personas based on a news personality, one or more personas based on a political personality, one or more personas based on a reality television personality, one or more personas based on a social media influencer, one or more personas based on a living celebrity, one or more personas based on a deceased celebrity, one or more personas based on a historical figure, one or more personas based on a fictionalization of a historical figure, one or more personas based on a character played by an actor or actress, one or more personas based on a bespoke character, or one or more personas simulating average humans in a geographical area within which the user is currently located or was previously residing, and/or the like.

In some instances, the user device 140 may each include, but is not limited to, one of a desktop computer, a laptop computer, a tablet computer, a smart phone, a mobile phone, or any suitable device capable of communicating with network(s) 155 or with computing system(s) 105a, gateway device 160, or other network devices within network(s) 155, or via any suitable device capable of communicating with at least one of the computing system(s) 105b, the user interactive system(s) 115b, the AI/ML system(s) 120b, the web portal 170, the provider server(s) 175a, the training server(s) 190a, and/or the therapy server(s) 195a, and/or the like, via a web-based portal (e.g., web portal 170, or the like), an application programming interface (“API”), a server, a software application (“app”), or any other suitable communications interface, or the like (not shown), over network(s) 155, 165a, and/or 165b, via gateway device 160, or the like.

In some cases, user 145 may include, without limitation, one of an individual, a customer of the provider, a student, an independent learner, a professional athlete, an amateur athlete, a craftsperson, an artist, a patient, a medical practitioner, an engineer, a scientist, a tradesperson or vocational worker, or a do-it-yourself (“DIY”) person, or the like. In some cases, location 150 may include, but is not limited to, one of a residential customer premises, a business customer premises, a corporate customer premises, an enterprise customer premises, an education facility customer premises, a medical facility customer premises, or a governmental customer premises, and/or the like.

According to some embodiments, network(s) 155, 165a, and/or 165b may each include, without limitation, one of a local area network (“LAN”), including, without limitation, a fiber network, an Ethernet network, a Token-Ring™ network, and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, and/or any other wireless protocol; and/or any combination of these and/or other networks. In a particular embodiment, the network(s) 155, 165a, and/or 165b may include an access network of the service provider (e.g., an Internet service provider (“ISP”)). In another embodiment, the network(s) 155, 165a, and/or 165b may include a core network of the service provider and/or the Internet.

In operation, at least one of computing system(s) 105, 105a, and/or 105b, user interactive system(s) 115, 115a, and/or 115b, and/or AI/ML system(s) 120a and/or 120b (collectively, “computing system” or the like) may generally perform the following tasks. In some examples, the computing system may cause at least one AI/ML-driven persona (e.g., AI/ML-driven persona(s) 125b, or the like) to interact with a user (e.g., user 145, or the like) via a UI (e.g., UI 135a or UI 135b, or the like), the interaction including a conversation between the at least one AI/ML-driven persona and the user. The computing system may analyze, using one of at least one AI/ML model (e.g., AI/ML model(s) 125a, or the like), the conversation to identify one or more goals of the conversation. The computing system may determine, using one of the at least one AI/ML model, a structure of the interaction with the user, based at least in part on the identified one or more goals of the conversation. The computing system may determine, using one of the at least one AI/ML model, one or more first parameters for the determined structure of the interaction with the user, the one or more first parameters defining conversational guardrails for steering the interaction away from conversational tangents and toward achieving at least one goal of the conversation. The computing system may generate, using one of the at least one AI/ML model, one or more first conversational threads configured to achieve the at least one goal of the conversation among the one or more goals of the conversation within the determined structure of the interaction with the user, in some cases, generating the one or more first conversational threads based on the one or more first parameters. The computing system may cause the at least one AI/ML-driven persona to continue the conversation with the user using the one or more first conversational threads to work toward achieving the at least one goal of the conversation.

In some cases, the computing system may map, using one of the at least one AI/ML model, a flow of the interaction with the user. Based on a determination that the flow of the interaction is moving away from achieving the at least one goal of the conversation, the computing system may determine, using one of the at least one AI/ML model, one or more second parameters for steering the interaction back toward achieving the at least one goal of the conversation. The computing system may generate, using one of the at least one AI/ML model, one or more second conversational threads configured to steer the interaction back toward achieving the at least one goal of the conversation, in some cases, generating the one or more second conversational threads based on the one or more second parameters. The computing system may cause the at least one AI/ML-driven persona to continue the conversation with the user using the one or more second conversational threads to steer the interaction back toward achieving the at least one goal of the conversation. In some embodiments, the approach taken by the computing system may be to obtain a goal or objective if there is none, to use strategy to do so, and to use tactics to achieve the goal.

According to some embodiments, the computing system may analyze, using one of the at least one AI/ML model, the interaction to identify one or more observable characteristics of the user. In some embodiments, the one or more observable characteristics may include (where applicable), but are not limited to, at least one of one or more speech patterns of the user, a language used by the user, whether the user has an accent, what accent the user has, whether English is a second language for the user, one or more non-verbal cues of the user, a demeanor of the user, a sentiment of the user, or an emotional state of the user, and/or the like.

In some embodiments, the computing system may access and analyze, using one of the at least one AI/ML model, stored information associated with the user to identify one or more conversation points. In some embodiments, the stored information may include (where applicable), but is not limited to, at least one of account information associated with the user, contact information associated with the user, billing information associated with the user, one or more contracts between the provider and the user, order history data associated with the user, previous interactions with the user, previous trouble tickets associated with the user, historical data associated with the user, demographic information about the user, personal information about the user, user-volunteered information regarding general interests of the user (e.g., the user may have mentioned that the user is a Denver Broncos fan, or the like), information regarding a market segment within which the user is classified, or societal information for a societal segment to which the user belongs, and/or the like. The computing system may cause the at least one AI/ML-driven persona to adapt by modifying the interaction with the user, based at least in part on at least one of the identified one or more observable characteristics of the user or the identified one or more conversation points, to enhance or improve the interaction with the user. In some cases, the computing system may utilize layered information, in order from a first layer to an N^{th} layer. For example, a first layer may include simple information about the user, while a second layer may include information about the user's account, and a third layer may include information about the market segment that the user is in, while a fourth layer may include societal information that is integrated into the AI/ML model, and a fifth layer may include personal information about the user, and so on. In some examples, the computing system may utilize per user database, training, and/or AI to combine lower-resolution per-company, per-market data, or the like, with higher-resolution per-user data. In a non-limiting example, per-user data may include “user has 2 children,” while per-company data may include “IBM is a large multinational corporation and people who work at IBM are familiar with processes,” and the computing system may utilize such data in a combined manner to make inferences about the user to facilitate or enhance interactions with the user.

In some examples, the computing system may identify and verify an identity of the user, in some cases, based at least in part on one or more of caller identification (“ID”) information, the account information associated with the user, a voiceprint of the user, two-factor authentication, or information provided by the user, and/or the like.

In some embodiments, the UI may be a visual-based UI. In such cases, the computing system, concurrent with the interaction with the user, may generate an avatar for each of the at least one AI/ML-driven persona. The computing system may display, within the UI, the avatar for each of the at least one AI/ML-driven persona. The computing system may animate, within the UI, the avatar in synchronization with the conversation with the user, where the interaction may further include the animation of the avatar. In some instances, each AI/ML-driven persona may have a set personality, the set personality including at least one of a set speech pattern, a set mannerism, a set command of one or more languages, a set accent, a set collection of non-verbal cues, or a set collection of emotional demeanors, and/or the like. In some examples, each of the interaction, the conversation, the one or more first conversational threads, and the animation of each AI/ML-driven persona may be performed in a manner consistent with the set personality of said AI/ML-driven persona.

In some cases, in response to user selection of a gamification mode, the computing system may perform the following: generating a visual representation of a list of the one or more goals; and in response to achieving a goal among the one or more goals, generating an animation of one or more of the at least one AI/ML-driven persona performing one or more actions to mark achievement of the goal, the one or more actions being performed in a manner consistent with the set personality of each of the one or more of the at least one AI/ML-driven persona. In some instances, one or more actions may include, but are not limited to, checking off the achieved goal, removing the achieved goal from the list, or marking the achieved goal as having been achieved, and/or the like.

In some examples, prior to the conversation or during a setup phase, the computing system may perform (where applicable) one of: (a) receiving a user selection of the at least one AI/ML-driven persona from a licensed set of AI/ML-driven personas among the plurality of AI/ML-driven personas with whom to interact; (b) identifying the user, and selecting the at least one AI/ML-driven persona from the licensed set of AI/ML-driven personas to match the user for interacting with the user, based on information regarding the identified user; (c) identifying the user, and selecting the at least one AI/ML-driven persona from the licensed set of AI/ML-driven personas to match a personality of a person whom the user is determined to respect or feel comfortable talking with for interacting with the user, based on information regarding the identified user; (d) setting a default set of AI/ML-driven personas from the licensed set of AI/ML-driven personas, wherein the default set of AI/ML-driven personas includes the at least one AI/ML-driven persona; or (e) randomly selecting the at least one AI/ML-driven persona from the licensed set of AI/ML-driven personas for interacting with the user; or the like.

In some instances, the computing system may perform (where applicable) at least one of: (1) adapting or adjusting, using one of the at least one AI/ML model, a personality of one or more of the at least one AI/ML-driven persona to mold to or match a determined personality of the user, where the personality of the user may be determined based on at least one of analysis of the interaction with the user, analysis of a previous interaction with the user, or known information about the user, and/or the like; (2) adapting or adjusting, using one of the at least one AI/ML model, one or more interaction characteristics of one or more of the at least one AI/ML-driven persona to match to a determined corresponding interaction characteristic of the user, the one or more interaction characteristics including at least one of speech pattern, language, accent, cultural mannerisms, cultural phraseology, general mannerisms, general phraseology, slang, jargon, or sentiment, and/or the like; (3) adapting or adjusting, using one of the at least one AI/ML model, a personality of one or more of the at least one AI/ML-driven persona to mold to or match a personality of a person whom the user is determined to respect or feel comfortable talking with, where the personality of the person whom the user is determined to respect or feel comfortable talking with is determined based on at least one of analysis of the interaction with the user, analysis of a previous interaction with the user, or known information about the user, and/or the like; or (4) adapting or adjusting, using one of the at least one AI/ML model, one or more interaction characteristics of one or more of the at least one AI/ML-driven persona to match to a determined corresponding interaction characteristic of the person whom the user is determined to respect or feel comfortable talking with, the one or more interaction characteristics including at least one of speech pattern, language, accent, cultural mannerisms, cultural phraseology, general mannerisms, general phraseology, slang, jargon, or sentiment, and/or the like; or the like.

According to some embodiments, the computing system, during the interaction with the user, may perform, using one of the at least one AI/ML model, at least one of continuous tone analysis or continuous sentiment analysis of the interaction with the user. After the interaction with the user, the computing system may update the at least one AI/ML model based on the at least one of continuous tone analysis or continuous sentiment analysis of the interaction with the user.

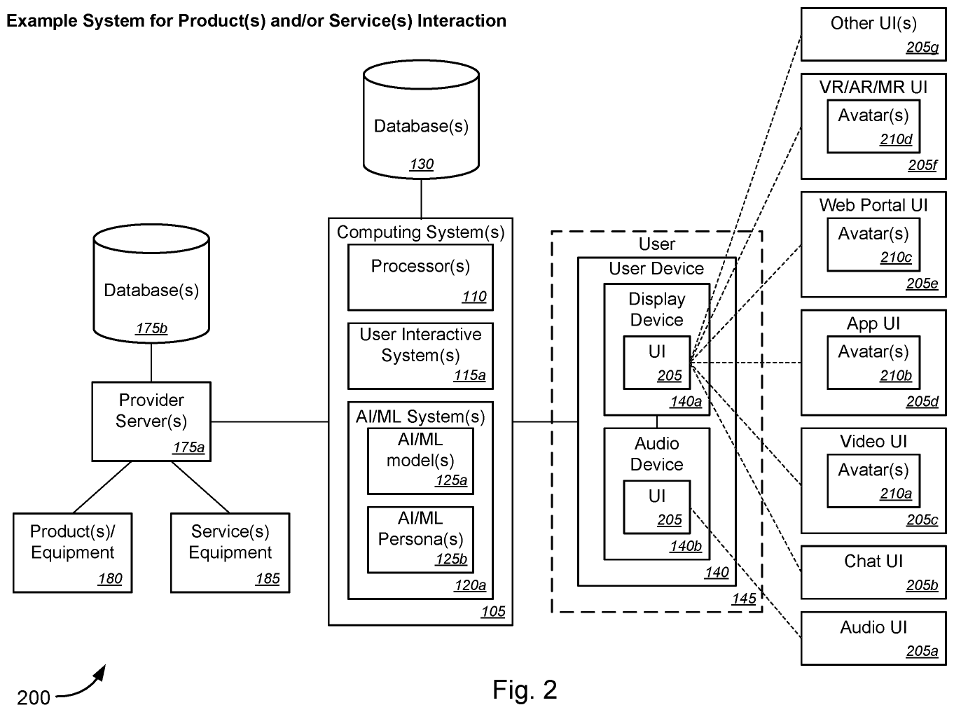

In some aspects, the computing system may perform the techniques, processes, and/or methods according to the non-limiting examples of specific use cases as described below with respect to FIGS. 2-5, or the like. These and other functions of the system 100 (and its components) are described in greater detail below with respect to FIGS. 2-5.

FIG. 2 is a schematic diagram illustrating a non-limiting example 200 of a system for implementing tailored synthetic personas with parameterized behaviors for engaging in a product(s) and/or service(s) interaction with a user, in accordance with various embodiments.

In the non-limiting example 200 of FIG. 2, computing system(s) 105 may include, without limitation, processor(s) 110, user interactive system(s) 115a, and AI/ML system(s) 120a, which may include AI/ML model(s) 125a and AI/ML-driven persona(s) 125b. Although user interactive system(s) 115a and AI/ML system(s) 120a are each shown in FIG. 2 to be integrated with, disposed within, or otherwise part of computing system(s) 105, user interactive system(s) and/or AI/ML system(s) that are external to computing system(s) 105 (in some cases, located within a network, such as network(s) 165a or 165b of FIG. 1, or the like) may alternatively or additionally be used. Computing system(s) 105 may communicatively couple with database(s) 130, user device 140, and provider server(s) 175a, in some cases, via one or more networks (e.g., network(s) 155, 165a, or 165b of FIG. 1, or the like). Provider server(s) 175a may communicatively couple with database(s) 175b, one or more products or equipment 180, and/or one or more service(s) equipment 185.

User device 140, which may be associated with or used by user 145, may include, but is not limited to, a display device 140a and/or an audio device 140b. Display device 140a may include, but is not limited to, at least one of a touchscreen display device, a non-touchscreen display device, a liquid crystal display (“LCD”) device, a light emitting diode (“LED”) display device, an organic LED (“oLED”) display device, and/or the like, while audio device 140b may include, but is not limited to, at least one of one or more microphones, one or more speakers, headphones (including acoustical or bone conduction, or the like), one or more earpieces, a headset, a telephone handset, and/or the like. In some cases, the display device 140a and/or audio device 140b may each include a UI(s) 205, including, but not limited to, at least one of an audio UI 205a, a chat UI 205b, a video UI 205c, an app UI 205d, a web portal UI 205e, a VR/AR/MR UI 205f, and/or other UI(s) 205g, which may include a telephone communication UI, a holographic UI, and/or the like. In some instances, unless otherwise specified, audio UI 205a may be used in conjunction with each of the chat UI 205b, the video UI 205c, the app UI 205d, the web portal UI 205e, the VR/AR/MR UI 205f, and/or the other UI(s) 205g, or the like. In some examples, for the video-based UIs, avatars 210 of the AI/ML personas 125 may be generated, displayed, and animated within the UIs 205. For instance, avatar(s) 210a, 210b, 210c, and 210d may be generated, displayed, and animated within the video UI 205c, the app UI 205d, the web portal UI 205e, and the VR/AR/MR UI 205f, respectively.

In some embodiments, computing system(s) 105, processor(s) 110, user interactive system(s) 115a, AI/ML system(s) 120a, AI/ML model(s) 125a, AI/ML-driven persona(s) 125b, database(s) 130, UI 205 and 205a-205g, user device 140, user 145, provider server(s) 175a, database(s) 175b, one or more products or equipment 180, and one or more service(s) equipment 185 of system 200 of FIG. 2 may correspond to computing system(s) 105, 105a, and/or 105b, processor(s) 110, user interactive system(s) 115, 115a, and/or 115b, AI/ML system(s) 120, 120a, and/or 120b, AI/ML model(s) 125a, AI/ML-driven persona(s) 125b, database(s) 130b and/or 130b, UI 135a and/or 135b, user device 140, user 145, provider server(s) 175a, database(s) 175b, one or more products or equipment 180, and one or more service(s) equipment 185 of system 100 of FIG. 1, respectively, and the descriptions of these components of system 100 are applicable to the corresponding components of system 200, respectively.

Further to the general tasks and operations that may be performed, as described above with respect to FIG. 1, the computing system may also perform the one or more of the following tasks. The computing system may cause at least one AI/ML-driven persona (e.g., AI/ML-driven persona(s) 125b, or the like) to interact with a user (e.g., user 145, or the like) via a UI (e.g., UI 205 or one or more of UIs 205a-205g, or the like), the interaction including a conversation between the at least one AI/ML-driven persona and the user. The computing system may analyze, using one of at least one AI/ML model (e.g., AI/ML model(s) 125a, or the like), the conversation to identify one or more goals of the conversation related to at least one of one or more products (e.g., one or more products or equipment 180, or the like) or one or more services provided (e.g., one or more service(s) equipment 185, or the like) by a provider (e.g., the provider associated with provider server(s) 175a, or the like). The computing system may determine, using one of the at least one AI/ML model, a structure of the interaction with the user, based at least in part on the identified one or more goals of the conversation. The computing system may determine, using one of the at least one AI/ML model, one or more first parameters for the determined structure of the interaction with the user, the one or more first parameters defining conversational guardrails for steering the interaction away from conversational tangents and toward achieving at least one goal of the conversation. The computing system may generate, using one of the at least one AI/ML model, one or more first conversational threads configured to achieve the at least one goal of the conversation among the one or more goals of the conversation, related to the at least one of the one or more products or the one or more services, within the determined structure of the interaction with the user, in some cases, generating the one or more first conversational threads based on the one or more first parameters. The computing system may cause the at least one AI/ML-driven persona to continue the conversation with the user using the one or more first conversational threads to work toward achieving the at least one goal of the conversation.

According to some embodiments, the one or more goals of the conversion may include, but are not limited to, at least one of purchasing the one or more products, ordering the one or more services, answering one or more questions regarding at least one product among the one or more products, answering one or more questions regarding at least one service among the one or more services, learning how to use at least one product among the one or more products, learning how to use at least one service among the one or more services, troubleshooting at least one issue associated with at least one product among the one or more products, troubleshooting at least one issue associated with at least one service among the one or more services, returning the one or more products, or ending the one or more services, and/or the like.

Merely by way of example, in some cases, the one or more products may include, without limitation, one or more of a telephone, a modem, a router, a customer premises equipment (“CPE”), an Ethernet circuit, a network device, a server, a consumer product, an electronic device, sporting goods, office equipment, a home appliance, a media recording device, a media player, a user device, clothing, footwear, a vehicle, or a building, and/or the like. In some examples, the one or more services may include, but are not limited to, one or more of electricity utility service, water utility service, trash and recycling pickup service, telephone service, cellular phone service, satellite telephone service, digital subscriber line (“DSL”) service, Internet service, Ethernet service, optical fiber Internet service, satellite Internet service, streaming media service, downloadable media service, cable television service, or satellite television service, and/or the like.

In some embodiments, concurrent with the interaction with the user, the computing system may cause the at least one AI/ML-driven persona to investigate a status of the at least one of the one or more products or the one or more services; may determine whether the computing system is capable of remotely addressing one or more issues with the at least one of the one or more products or the one or more services; and based on a determination that the computing system is capable of remotely addressing one or more issues with the at least one of the one or more products or the one or more services, may generate a conversational message indicating that the at least one AI/ML-driven persona is able to remotely address the one or more issues with the at least one of the one or more products or the one or more services and will proceed to do so, and may initiate one or more processes to remotely address the one or more issues with the at least one of the one or more products or the one or more services. In some examples, determining whether the computing system is capable of remotely addressing the one or more issues with the at least one of the one or more products or the one or more services may include remotely accessing one or more systems associated with the at least one of the one or more products or the one or more services (e.g., provider server(s) 175a, or the like), and remotely running one or more tests on the one or more systems (e.g., provider server(s) 175a, or the like), the one or more tests including a connectivity and control test, or the like.

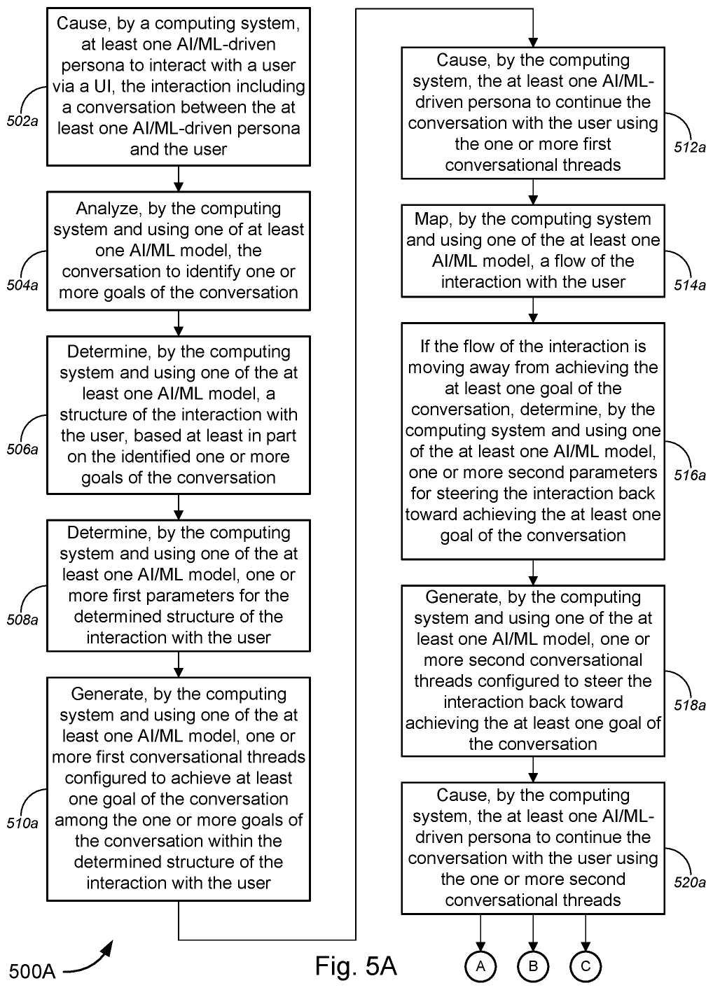

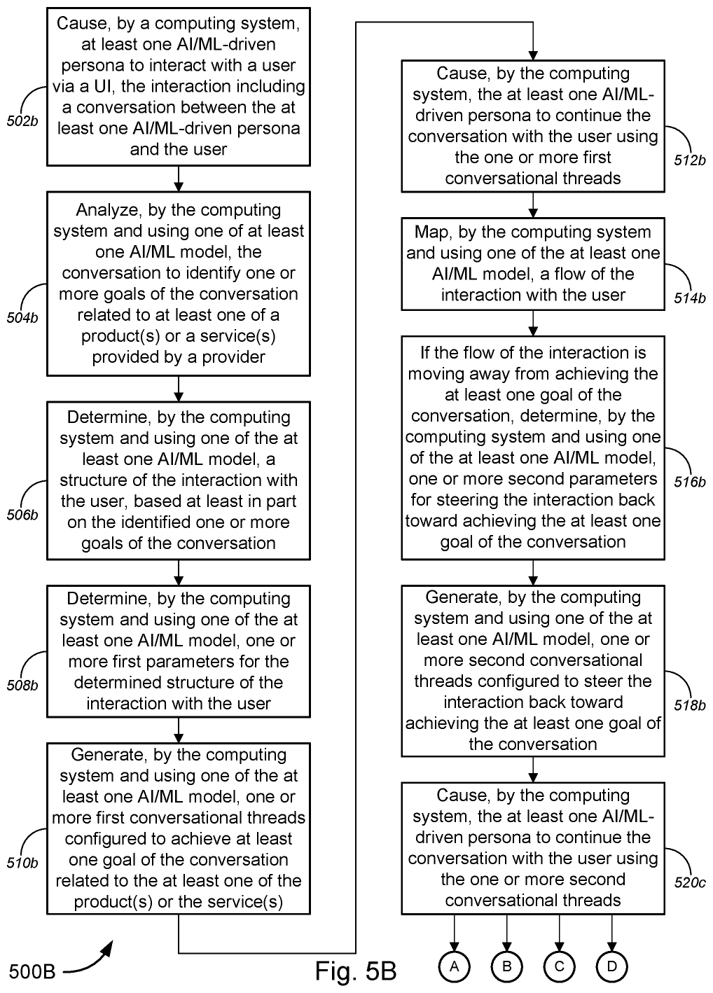

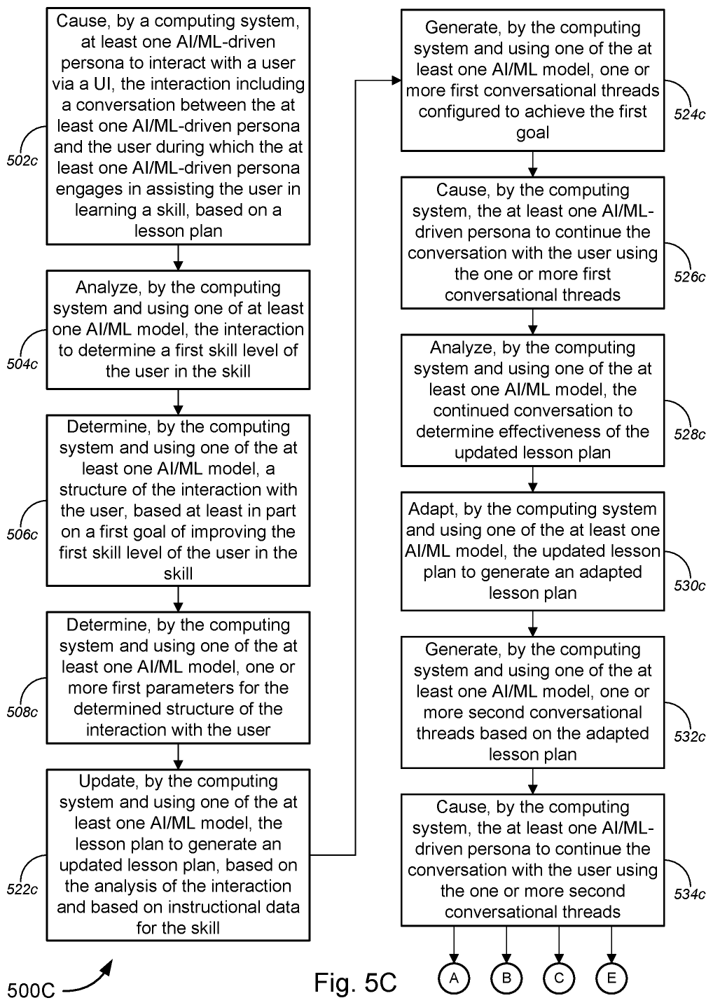

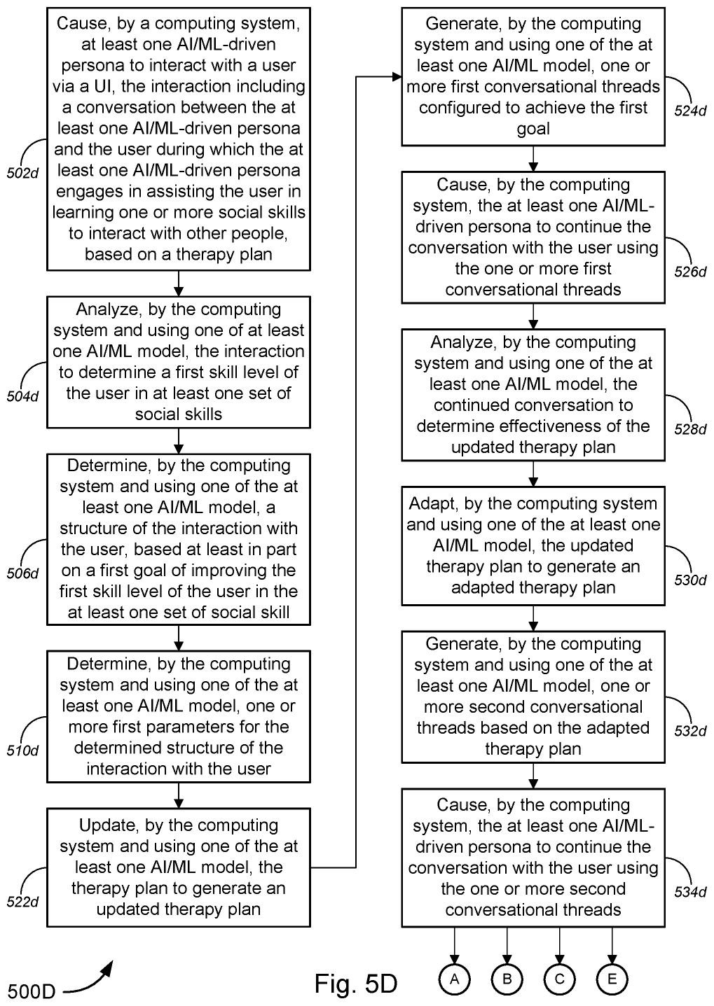

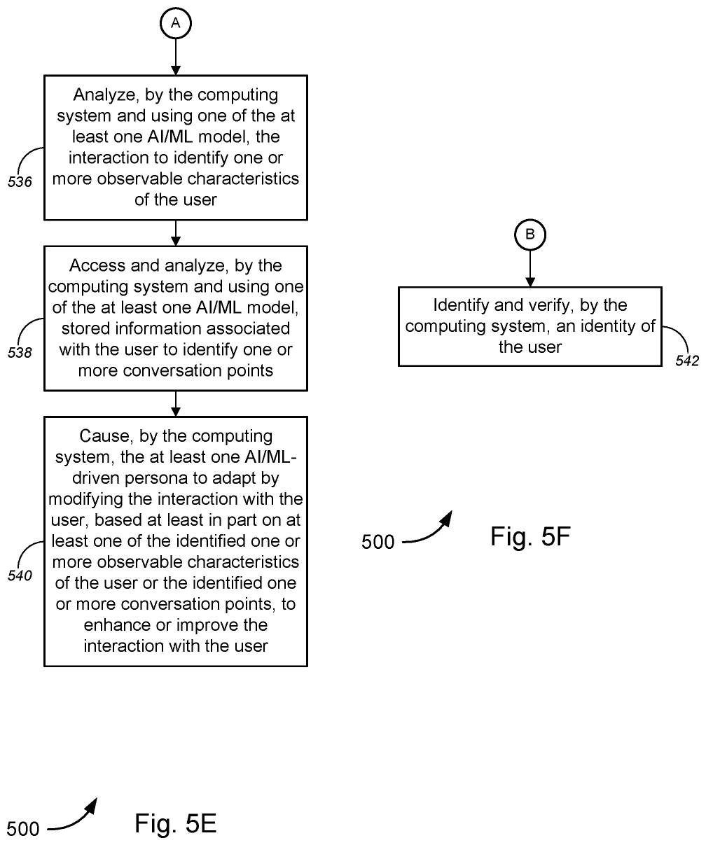

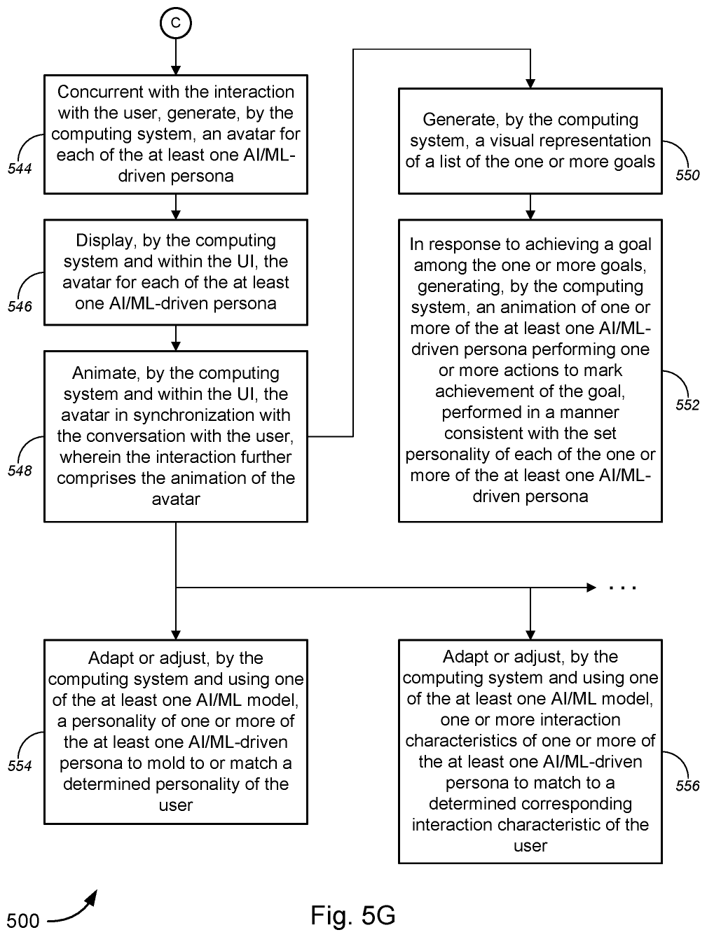

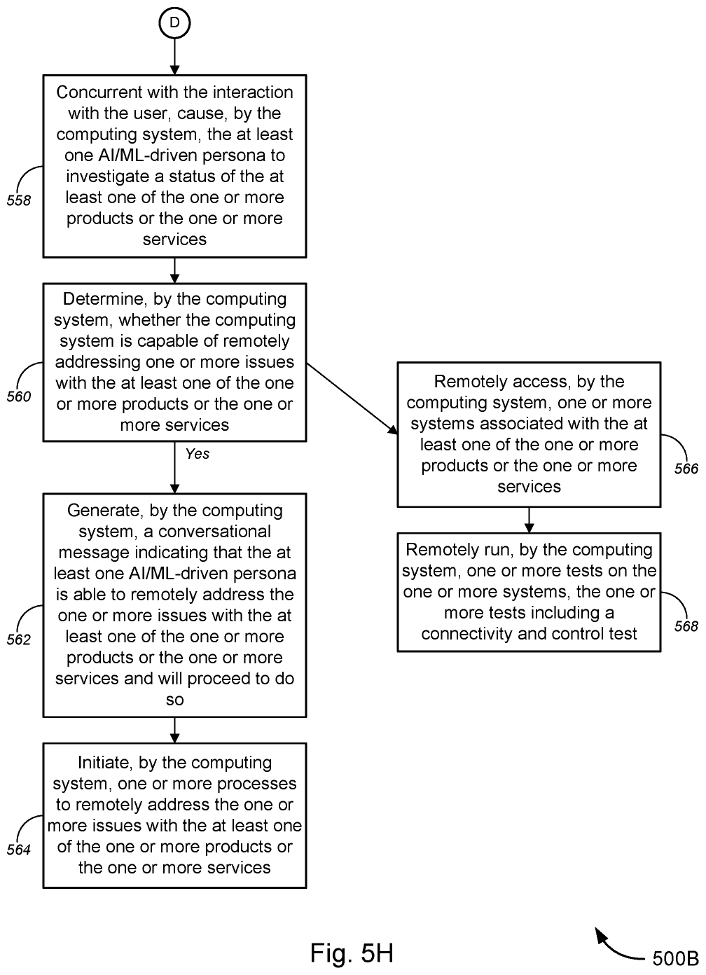

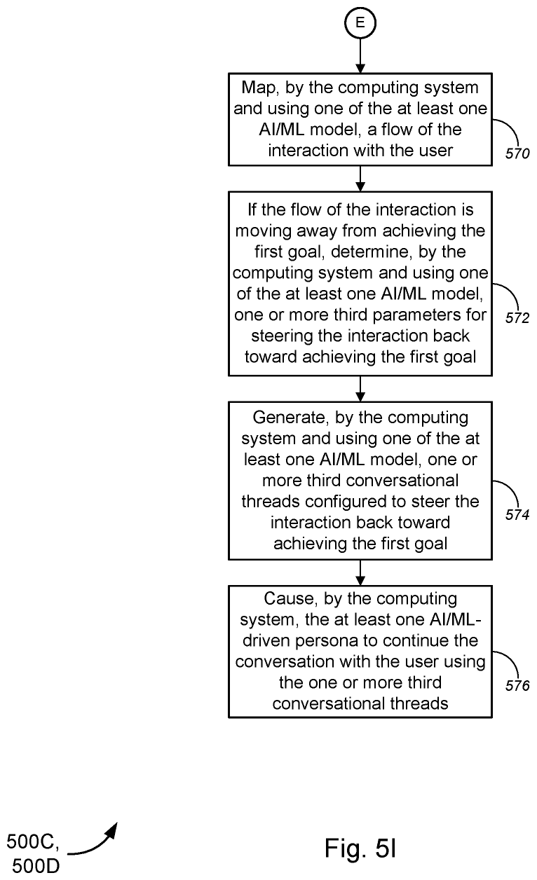

These and other functions of the example 200 (and its components) are described in greater detail herein with respect to FIGS. 1 and 5.

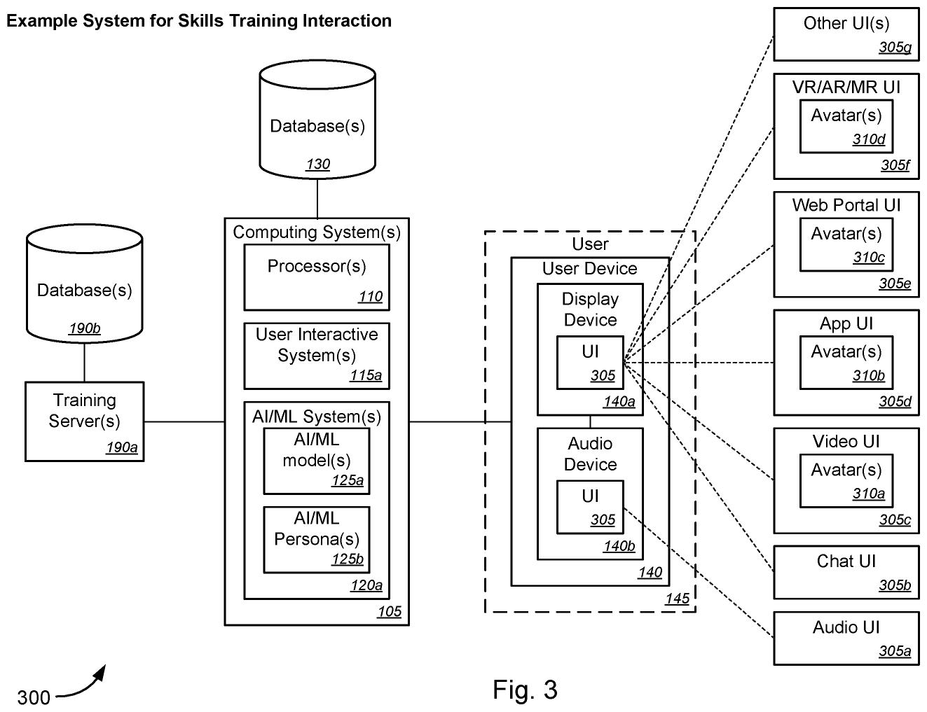

FIG. 3 is a schematic diagram illustrating a non-limiting example 300 of a system for implementing tailored synthetic personas with parameterized behaviors for engaging in a skills training interaction with a user, in accordance with various embodiments.

In the non-limiting example 300 of FIG. 3, computing system(s) 105 may include, without limitation, processor(s) 110, user interactive system(s) 115a, and AI/ML system(s) 120a, which may include AI/ML model(s) 125a and AI/ML-driven persona(s) 125b. Although user interactive system(s) 115a and AI/ML system(s) 120a are each shown in FIG. 3 to be integrated with, disposed within, or otherwise part of computing system(s) 105, user interactive system(s) and/or AI/ML system(s) that are external to computing system(s) 105 (in some cases, located within a network, such as network(s) 165a or 165b of FIG. 1, or the like) may alternatively or additionally be used. Computing system(s) 105 may communicatively couple with database(s) 130, user device 140, and training server(s) 190a, in some cases, via one or more networks (e.g., network(s) 155, 165a, or 165b of FIG. 1, or the like). Training server(s) 190a may communicatively couple with database(s) 190b.

User device 140, which may be associated with or used by user 145, may include, but is not limited to, a display device 140a and/or an audio device 140b. Display device 140a may include, but is not limited to, at least one of a touchscreen display device, a non-touchscreen display device, a liquid crystal display (“LCD”) device, a light emitting diode (“LED”) display device, an organic LED (“oLED”) display device, and/or the like, while audio device 140b may include, but is not limited to, at least one of one or more microphones, one or more speakers, headphones (including acoustical or bone conduction, or the like), one or more earpieces, a headset, a telephone handset, and/or the like. In some cases, the display device 140a and/or audio device 140b may each include a UI(s) 305, including, but not limited to, at least one of an audio UI 305a, a chat UI 305b, a video UI 305c, an app UI 305d, a web portal UI 305e, a VR/AR/MR UI 305f, and/or other UI(s) 305g, which may include a telephone communication UI, a holographic UI, and/or the like. In some instances, unless otherwise specified, audio UI 305a may be used in conjunction with each of the chat UI 305b, the video UI 305c, the app UI 305d, the web portal UI 305e, the VR/AR/MR UI 305f, and/or the other UI(s) 305g, or the like. In some examples, for the video-based UIs, avatars 310 of the AI/ML personas 125 may be generated, displayed, and animated within the UIs 305. For instance, avatar(s) 310a, 310b, 310c, and 310d may be generated, displayed, and animated within the video UI 305c, the app UI 305d, the web portal UI 305e, and the VR/AR/MR UI 305f, respectively.

In some embodiments, computing system(s) 105, processor(s) 110, user interactive system(s) 115a, AI/ML system(s) 120a, AI/ML model(s) 125a, AI/ML-driven persona(s) 125b, database(s) 130, UI 305 and 305a-205g, user device 140, user 145, training server(s) 190a, and database(s) 190b of system 300 of FIG. 3 may correspond to computing system(s) 105, 105a, and/or 105b, processor(s) 110, user interactive system(s) 115, 115a, and/or 115b, AI/ML system(s) 120, 120a, and/or 120b, AI/ML model(s) 125a, AI/ML-driven persona(s) 125b, database(s) 130b and/or 130b, UI 135a and/or 135b, user device 140, user 145, training server(s) 190a, and database(s) 190b of system 100 of FIG. 1, respectively, and the descriptions of these components of system 100 are applicable to the corresponding components of system 300, respectively.

Further to the general tasks and operations that may be performed, as described above with respect to FIG. 1, the computing system may also perform the one or more of the following tasks. The computing system may cause at least one AI/ML-driven persona (e.g., AI/ML-driven persona(s) 125b, or the like) to interact with a user (e.g., user 145, or the like) via a UI (e.g., UI 305 or one or more of UIs 305a-305g, or the like), the interaction including a conversation between the at least one AI/ML-driven persona and the user during which the at least one AI/ML-driven persona engages in assisting the user in learning a skill. In some examples, assisting the user in learning the skill may be based on a lesson plan that, in some cases, may be generated based on instructional data for the skill that may be stored in a database (e.g., database(s) 190b, or the like). The computing system may analyze, using one of at least one AI/ML model (e.g., AI/ML model(s) 125a, or the like), the interaction to determine a first skill level of the user in the skill, the first skill level including at least one of a level of knowledge of the skill that the user possesses, a level of understanding of the skill by the user, or a level of ability of the user to apply the skill under one or more conditions, and/or the like. The computing system may determine, using one of the at least one AI/ML model, a structure of the interaction with the user, based at least in part on a first goal of improving the first skill level of the user in the skill. The computing system may determine, using one of the at least one AI/ML model, one or more first parameters for the determined structure of the interaction with the user, the one or more first parameters defining conversational guardrails for steering the interaction away from conversational tangents and toward achieving the first goal. The computing system may update, using one of the at least one AI/ML model, the lesson plan to generate an updated lesson plan, in some cases, based on the analysis of the interaction and/or based on instructional data for the skill.

The computing system may generate, using one of the at least one AI/ML model, one or more first conversational threads configured to achieve the first goal of improving the first skill level of the user in the skill, based on the analysis of the interaction and based on instructional data for the skill that is accessible from the database (e.g., database(s) 190b, or the like), in some cases, generating the one or more first conversational threads may be further based on the one or more first parameters and/or based on the updated lesson plan. The computing system may cause the at least one AI/ML-driven persona to continue the conversation with the user using the one or more first conversational threads to work toward achieving the first goal. The computing system may analyze, using one of the at least one AI/ML model, the continued conversation to determine effectiveness of the updated lesson plan, in some cases, by determining whether there are any changes to the first skill level of the user in the skill, after use of the one or more first conversational threads. The computing system may adapt, using one of the at least one AI/ML model, the updated lesson plan to generate an adapted lesson plan, in some cases, based on the analysis of the continued conversation and/or based on the instructional data for the skill. The computing system may generate using one of the at least one AI/ML model, one or more third conversational threads based on the adapted lesson plan, and may cause the at least one AI/ML-driven persona to continue the conversation with the user using the one or more third conversational threads.

According to some embodiments, the skill may be among a plurality of skills including, but not limited to, at least one of skill in a language among a plurality of languages (including, without limitation, English, Mandarin, Hindi, Spanish, French, Arabic, Bengali, Russian, Portuguese, Indonesian, etc.), skill in understanding a culture among a plurality of cultures, skill in living in nature, skill in living in small population centers, skill in living in large population centers, skill in living in rural areas, skill in living in cities, skill in navigating tourist areas, skill in an elementary school course, skill in a middle school course, skill in a high school course, skill in a college course, skill in a university course, skill in a vocational course, skill in mathematics, skill in physics, skill in chemistry, skill in biology, skill in sociology, skill in philosophy, skill in psychology, skill in computer programming, skill in literary studies, skill in linguistics, skill in writing, skill in civics or social studies, skill in historical studies, skill in geography, skill in geology, skill in engineering, skill in educating others, skill in training animals, skill in home repairs, skill in vehicle repairs, skill in appliance repairs, skill in computer repairs, skill in mobile device repairs, skill in assembling a consumer product, skill in construction, skill in driving, skill in sports activity, skill in recreational activity, skill in card games, skill in board games, skill in trivia games, skill in cooking, skill in butchering meats, skill in cleaning, skill in crafting, skill in carpentry, skill in metalworking, skill in smithing, skill in artistic forms, skill in painting, skill in first aid, skill in navigation, skill in buying, skill in selling, skill in bartering, skill in negotiation, skill in trading, skill in marketing, skill in accounting, skill in business development, skill in public speaking, skill in giving presentations, skill in communication, skill in management, or skill in leadership, and/or the like.

In some embodiments, the computing system may perform at least one of: (A)(1) using a first set of AI/ML-driven personas to assist the user in learning a first skill among the plurality of skills, and (A)(2) using a second set of AI/ML-driven personas that is different from the first set of AI/ML-driven personas to assist the user in learning a second skill among the plurality of skills that is different from the first skill; or (B)(1) using a first set of learning strategies among a plurality of learning strategies for assisting the user in learning the first skill, and (B)(2) using a second set of learning strategies among the plurality of learning strategies that is different from the first set of learning strategies for assisting the user in learning the second skill. In some examples, the plurality of learning strategies may include rote learning, learning using flash cards or virtual flash cards, learning by interaction, learning by practice, auditory learning, visual learning, or learning using a combination of two or more of said learning strategies; and/or the like.

In some instances, the skill may be skill in a first language among a plurality of languages (including, without limitation, English, Mandarin, Hindi, Spanish, French, Arabic, Bengali, Russian, Portuguese, Indonesian, etc.). In such cases, analyzing the conversation or the continued conversation may include the computing system performing at least one of: analyzing, using one of at least one AI/ML model, the interaction to determine at least one of a breadth of a breadth of listening vocabulary, a depth of listening vocabulary, a breadth of speaking vocabulary, a depth of speaking vocabulary, reading vocabulary, a depth of reading vocabulary, a breadth of writing vocabulary, or a depth of writing vocabulary that the user has in the first language, and/or the like; analyzing, using one of at least one AI/ML model, the interaction to determine the user's proficiency and understanding of grammar in the first language, the grammar including at least one of tense, number, noun classes, locative relations, syntax, grammatical structure, or grammatical gender, and/or the like; analyzing, using one of at least one AI/ML model, the interaction to determine the user's pronunciation of words in the first language; analyzing, using one of at least one AI/ML model, the interaction to determine an optimal learning strategy among a plurality of learning strategies for the user to learn the first language, the plurality of learning strategies including learning by flash cards or virtual flash cards, learning by usage in sentences, learning by vocabulary drills, learning by exposure to particular words in different contexts, learning by language immersion, learning by interaction, learning by reading sentences or passages, learning by speaking sentences, learning by listening to conversations (e.g., between two or more AI/ML-driven personas, or the like), auditory learning, visual learning, learning by translating to a second language among the plurality of languages that is different from the first language, or learning using a combination of two or more of said learning strategies, and/or the like; analyzing, using one of at least one AI/ML model, the interaction to determine how quickly the user interacts or responds; or analyzing, using one of at least one AI/ML model, the interaction to determine which AI/ML-driven persona or which combination of two or more AI/ML-driven personas best assists the user in learning the first language; and/or the like.

In some cases, updating the lesson plan or adapting the updated lesson plan may include the computing system performing at least one of: changing one or more first AI/ML-driven personas among the at least one AI/ML-driven persona to one or more second AI/ML-driven persona among the at least one AI/ML-driven persona; changing at least one of a voice or speech pattern, a tone, an accent, a pitch, a cadence, or a gender of the at least one AI/ML-driven persona, and/or the like; changing at least one of a time of day, a time of week, or a time of month that the lesson plan or the updated lesson plan is implemented, and/or the like; changing a pace of the lesson plan or the updated lesson plan; or changing a learning strategy among a plurality of learning strategies from a first learning strategy to a second learning strategy, the plurality of learning strategies including rote learning, learning using flash cards or virtual flash cards, learning by interaction, learning by practice, auditory learning, visual learning, or learning using a combination of two or more of said learning strategies, and/or the like; and/or the like.

These and other functions of the example 300 (and its components) are described in greater detail herein with respect to FIGS. 1 and 5.

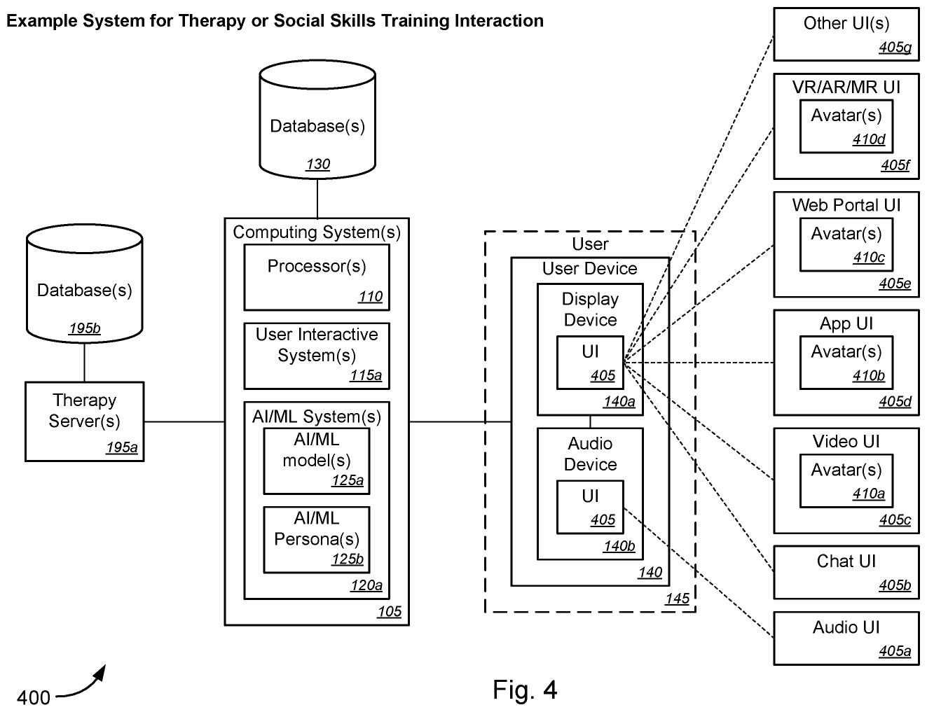

FIG. 4 is a schematic diagram illustrating a non-limiting example 400 of a system for implementing tailored synthetic personas with parameterized behaviors for engaging in a therapy or social skills training interaction with a user, in accordance with various embodiments.

In the non-limiting example 400 of FIG. 4, computing system(s) 105 may include, without limitation, processor(s) 110, user interactive system(s) 115a, and AI/ML system(s) 120a, which may include AI/ML model(s) 125a and AI/ML-driven persona(s) 125b. Although user interactive system(s) 115a and AI/ML system(s) 120a are each shown in FIG. 4 to be integrated with, disposed within, or otherwise part of computing system(s) 105, user interactive system(s) and/or AI/ML system(s) that are external to computing system(s) 105 (in some cases, located within a network, such as network(s) 165a or 165b of FIG. 1, or the like) may alternatively or additionally be used. Computing system(s) 105 may communicatively couple with database(s) 130, user device 140, and therapy server(s) 195a, in some cases, via one or more networks (e.g., network(s) 155, 165a, or 165b of FIG. 1, or the like). Therapy server(s) 195a may communicatively couple with database(s) 195b.

User device 140, which may be associated with or used by user 145, may include, but is not limited to, a display device 140a and/or an audio device 140b. Display device 140a may include, but is not limited to, at least one of a touchscreen display device, a non-touchscreen display device, a liquid crystal display (“LCD”) device, a light emitting diode (“LED”) display device, an organic LED (“OLED”) display device, and/or the like, while audio device 140b may include, but is not limited to, at least one of one or more microphones, one or more speakers, headphones (including acoustical or bone conduction, or the like), one or more earpieces, a headset, a telephone handset, and/or the like. In some cases, the display device 140a and/or audio device 140b may each include a UI(s) 405, including, but not limited to, at least one of an audio UI 405a, a chat UI 405b, a video UI 405c, an app UI 405d, a web portal UI 405e, a VR/AR/MR UI 405f, and/or other UI(s) 405g, which may include a telephone communication UI, a holographic UI, and/or the like. In some instances, unless otherwise specified, audio UI 405a may be used in conjunction with each of the chat UI 405b, the video UI 405c, the app UI 405d, the web portal UI 405e, the VR/AR/MR UI 405f, and/or the other UI(s) 405g, or the like. In some examples, for the video-based UIs, avatars 410 of the AI/ML personas 125 may be generated, displayed, and animated within the UIs 405. For instance, avatar(s) 410a, 410b, 410c, and 410d may be generated, displayed, and animated within the video UI 405c, the app UI 405d, the web portal UI 405e, and the VR/AR/MR UI 405f, respectively.

In some embodiments, computing system(s) 105, processor(s) 110, user interactive system(s) 115a, AI/ML system(s) 120a, AI/ML model(s) 125a, AI/ML-driven persona(s) 125b, database(s) 130, UI 405 and 405a-205g, user device 140, user 145, therapy server(s) 195a, and database(s) 195b of system 400 of FIG. 4 may correspond to computing system(s) 105, 105a, and/or 105b, processor(s) 110, user interactive system(s) 115, 115a, and/or 115b, AI/ML system(s) 120, 120a, and/or 120b, AI/ML model(s) 125a, AI/ML-driven persona(s) 125b, database(s) 130b and/or 130b, UI 135a and/or 135b, user device 140, user 145, therapy server(s) 195a, and database(s) 195b of system 100 of FIG. 1, respectively, and the descriptions of these components of system 100 are applicable to the corresponding components of system 400, respectively.

Further to the general tasks and operations that may be performed, as described above with respect to FIG. 1, the computing system may also perform the one or more of the following tasks. The computing system may cause at least one AI/ML-driven persona (e.g., AI/ML-driven persona(s) 125b, or the like) to interact with a user (e.g., user 145, or the like) via a UI (e.g., UI 405 or one or more of UIs 405a-405g, or the like), the interaction including a conversation between the at least one AI/ML-driven persona and the user during which the at least one AI/ML-driven persona engages in assisting the user in learning one or more social skills to interact with other people. In some examples, assisting the user in learning the one or more social skills may be based on a therapy plan that, in some cases, may be generated based on social or behavioral therapy data for the at least one set of social skills that may be stored in a database (e.g., database(s) 195b, or the like). The computing system may analyze, using one of at least one AI/ML model (e.g., AI/ML model(s) 125a, or the like), the interaction to determine a first skill level of the user in at least one set of social skills among the one or more social skills, the first skill level comprising a level of competence in the at least one set of social skill that the user possesses. The computing system may determine, using one of the at least one AI/ML model, a structure of the interaction with the user, based at least in part on a first goal of developing the first skill level of the user in the at least one set of social skill. The computing system may determine, using one of the at least one AI/ML model, one or more first parameters for the determined structure of the interaction with the user, the one or more first parameters defining conversational guardrails for steering the interaction away from conversational tangents and toward achieving the first goal. The computing system may update, using one of the at least one AI/ML model, the therapy plan to generate an updated therapy plan, in some cases, based on the analysis of the interaction and/or based on social or behavioral therapy data for the at least one set of social skills.

The computing system may generate, using one of the at least one AI/ML model, one or more first conversational threads configured to achieve the first goal of developing the first skill level of the user in the at least one set of social skill, based on the analysis of the interaction and based on social or behavioral therapy data for the at least one set of social skills that is accessible from the database (e.g., database(s) 195b, or the like), in some cases, generating the one or more first conversational threads may be further based on the one or more first parameters and/or based on the updated therapy plan. The computing system may cause the at least one AI/ML-driven persona to continue the conversation with the user using the one or more first conversational threads to work toward achieving the first goal. The computing system may analyze, using one of the at least one AI/ML model, the continued conversation to determine effectiveness of the updated therapy plan, by determining whether there are any changes to the first skill level of the user in the at least one set of social skills, after use of the one or more first conversational threads. The computing system may adapt, using one of the at least one AI/ML model, the updated therapy plan to generate an adapted therapy plan, in some cases, based on the analysis of the continued conversation and/or based on the social or behavioral therapy data for the at least one set of social skills. The computing system may generate using one of the at least one AI/ML model, one or more third conversational threads based on the adapted therapy plan, and may cause the at least one AI/ML-driven persona to continue the conversation with the user using the one or more third conversational threads.

According to some embodiments, the one or more social skills may include, without limitation, at least one of social coordination skills, mentoring skills, negotiation skills, persuasion skills, psychosocial service orientation skills, social perceptiveness skills, active listening skills, delegation skills, decision-making skills, problem-solving skills, creative thinking skills, critical thinking skills, communication skills, interpersonal skills, self-awareness skills, empathy skills, assertiveness skills, equanimity skills, psychological resilience skills, or coping skills, and/or the like.

In some embodiments, the computing system may perform at least one of: (A)(1) using a first set of AI/ML-driven personas to assist the user in learning a first set of social skills among the one or more social skills, and (A)(2) using a second set of AI/ML-driven personas that is different from the first set of AI/ML-driven personas to assist the user in learning a second set of social skill among the one or more social skills that is different from the first set of social skills; or (B)(1) using a first set of social training strategies among a plurality of social training strategies for assisting the user in learning the first set of social skills, and (B)(2) using a second set of social training strategies among the plurality of social training strategies that is different from the first set of social training strategies for assisting the user in learning the second set of social skills. In some examples, the plurality of social training strategies may include encouragement of use of at least one of self-reflection, meditation, social setting simulation, empathetic interaction, behavioral adjustment, conversation training, psychotherapy treatment, cognitive behavioral therapy (“CBT”) or computerized CBT, dialectical behavior therapy, simulated hypnotherapy, art therapy, or learning using a combination of two or more of said social training strategies, and/or the like.

In some embodiments, the at least one set of social skills may be a skill for interacting with multiple people at a time. In such cases, analyzing the conversation or the continued conversation may include the computing system performing at least one of: analyzing, using one of at least one AI/ML model, the interaction to determine a level of social anxiety that the user possesses; or analyzing, using one of at least one AI/ML model, the interaction to identify one or more triggers for the user's discomfort arising from social anxiety. In some instances, the computing system may perform at least one of: causing a plurality of different AI/ML-driven personas to simulate a crowd of people whose number is determined to be one of the identified one or more triggers; implementing a therapy plan in which the user is introduced to a different sets of AI/ML-driven personas, each successive set increasing in number of different AI/ML-driven personas as the user becomes more comfortable interacting with each set of AI/ML-driven personas, to assist the user in becoming desensitized to crowd triggers for social anxiety; or simulating a social setting in which the user is planning to speak, by filling the simulated social setting with a plurality of different AI/ML-driven personas that are caused to interact with the user within the context of the social setting.

According to some embodiments, updating the therapy plan or adapting the updated therapy plan may include the computing system performing at least one of: changing one or more first AI/ML-driven personas among the at least one AI/ML-driven persona to one or more second AI/ML-driven persona among the at least one AI/ML-driven persona; changing at least one of a voice or speech pattern, a tone, an accent, a pitch, a cadence, or a gender of the at least one AI/ML-driven persona, and/or the like; changing at least one of a time of day, a time of week, or a time of month, and/or the like, that the therapy plan or the updated therapy plan is implemented; changing a pace of the therapy plan or the updated therapy plan; or changing a set of social training strategies among a plurality of social training strategies from a first set of social training strategies to a second set of social training strategies. In some examples, the plurality of social training strategies may include encouragement of use of at least one of self-reflection, meditation, social setting simulation, empathetic interaction, behavioral adjustment, conversation training, psychotherapy treatment, cognitive behavioral therapy (“CBT”) or computerized CBT, dialectical behavior therapy, simulated hypnotherapy, art therapy, or learning using a combination of two or more of said social training strategies, and/or the like.

These and other functions of the example 400 (and its components) are described in greater detail herein with respect to FIGS. 1 and 5.

FIGS. 5A-5I (collectively, “FIG. 5”) are flow diagrams illustrating a method(s) 500, 500A, 500B, 500C, and/or 500D for implementing tailored synthetic personas with parameterized behaviors, in accordance with various embodiments. Method 500A is directed to causing an artificial intelligence (“AI”)/machine learning (“ML”)-driven persona(s) to generally engage in conversational interactions with a user, while method 500B is directed to causing an AI/ML-driven persona(s) to engage in conversational interactions with a user to discuss a product(s) and/or service(s) provided by a provider. Method 500C is directed to causing an AI/ML-driven persona(s) to engage in assisting a user in learning a skill during conversational interactions, while method 500D is directed to causing an AI/ML-driven persona(s) to engage in assisting a user in learning one or more social skills to interact with other people during conversational interactions, and method 500 is directed to processes that may be applicable to one or more of methods 500A, 500B, 500C, and/or 500D.

Method 500A of FIG. 5A continues onto FIG. 5E following the circular marker denoted, “A.” Alternatively, or additionally, method 500A of FIG. 5A continues onto FIG. 5F following the circular marker denoted, “B.” Alternatively, or additionally, method 500A of FIG. 5A continues onto FIG. 5G following the circular marker denoted, “C.” Method 500B of FIG. 5B continues onto FIG. 5E following the circular marker denoted, “A.” Alternatively, or additionally, method 500B of FIG. 5B continues onto FIG. 5F following the circular marker denoted, “B.” Alternatively, or additionally, method 500B of FIG. 5B continues onto FIG. 5G following the circular marker denoted, “C.” Alternatively, or additionally, method 500D of FIG. 5D continues onto FIG. 5H following the circular marker denoted, “D.” Method 500C of FIG. 5C and/or method 500D of FIG. 5D continues onto FIG. 5E following the circular marker denoted, “A.” Alternatively, or additionally, method 500C of FIG. 5C and/or method 500D of FIG. 5D of FIG. 5B continues onto FIG. 5F following the circular marker denoted, “B.” Alternatively, or additionally, method 500C of FIG. 5C and/or method 500D of FIG. 5D continues onto FIG. 5G following the circular marker denoted, “C.” Alternatively, or additionally, method 500C of FIG. 5C and/or method 500D of FIG. 5D continues onto FIG. 5I following the circular marker denoted, “E.”

While the techniques and procedures are depicted and/or described in a certain order for purposes of illustration, it should be appreciated that certain procedures may be reordered and/or omitted within the scope of various embodiments. Moreover, while the method 500 illustrated by FIG. 5 can be implemented by or with (and, in some cases, are described below with respect to) the systems, examples, or embodiments 100, 200, 300, and 400 of FIGS. 1, 2, 3 and 4, respectively (or components thereof), such methods may also be implemented using any suitable hardware (or software) implementation. Similarly, while each of the systems, examples, or embodiments, in some cases, are described below with respect to) the systems, examples, or embodiments 100, 200, 300, and 400 of FIGS. 1, 2, 3 and 4, respectively (or components thereof), can operate according to the method 500 illustrated by FIG. 5 (e.g., by executing instructions embodied on a computer readable medium), the systems, examples, or embodiments, in some cases, are described below with respect to) the systems, examples, or embodiments 100, 200, 300, and 400 of FIGS. 1, 2, 3 and 4 can each also operate according to other modes of operation and/or perform other suitable procedures.

In the non-limiting embodiment of FIG. 5A, method 500A, at block 502a, may include causing, by a computing system, at least one artificial intelligence (“AI”)/machine learning (“ML”)-driven persona to interact with a user via a user interface (“UI”), the interaction including a conversation between the at least one AI/ML-driven persona and the user. At block 504a, method 500A may include analyzing, by the computing system and using one of at least one AI/ML model, the conversation to identify one or more goals of the conversation. Method 500A may further include, at block 506a, determining, by the computing system and using one of the at least one AI/ML model, a structure of the interaction with the user, based at least in part on the identified one or more goals of the conversation. Method 500A may further include determining, by the computing system and using one of the at least one AI/ML model, one or more first parameters for the determined structure of the interaction with the user, the one or more first parameters defining conversational guardrails for steering the interaction away from conversational tangents and toward achieving at least one goal of the conversation (block 508a). At block 510a, method 500A may include generating, by the computing system and using one of the at least one AI/ML model, one or more first conversational threads configured to achieve the at least one goal of the conversation among the one or more goals of the conversation within the determined structure of the interaction with the user, in some cases, generating the one or more first conversational threads based on the one or more first parameters. Method 500A may further include, at block 512a, causing, by the computing system, the at least one AI/ML-driven persona to continue the conversation with the user using the one or more first conversational threads to work toward achieving the at least one goal of the conversation.

Method 500A, at block 514a, may include, mapping, by the computing system and using one of the at least one AI/ML model, a flow of the interaction with the user. At block 516a, method 500A may include, based on a determination that the flow of the interaction is moving away from achieving the at least one goal of the conversation, determining, by the computing system and using one of the at least one AI/ML model, one or more second parameters for steering the interaction back toward achieving the at least one goal of the conversation. Method 500A may further include, at block 518a, generating, by the computing system and using one of the at least one AI/ML model, one or more second conversational threads configured to steer the interaction back toward achieving the at least one goal of the conversation, in some cases, generating the one or more second conversational threads based on the one or more second parameters. Method 500A may further include causing, by the computing system, the at least one AI/ML-driven persona to continue the conversation with the user using the one or more second conversational threads to steer the interaction back toward achieving the at least one goal of the conversation (block 520a).

In some embodiments, the computing system may include, without limitation, at least one of a server, an AI system, a ML system, an AI/ML system, a deep learning (“DL”) system, a user interactive system, a customer interface server, a cloud computing system, or a distributed computing system, and/or the like. In some cases, the UI may include, but is not limited to, one of a voice-only UI, a telephone communication UI, a video-only UI, a video with voice UI, a chat UI, a software application (“app”) UI, a holographic UI, a virtual reality (“VR”)-based UI, an augmented reality (“AR”)-based UI, a mixed reality (“MR”)-based UI, or a web-portal-based UI, and/or the like.

According to some embodiments, the at least one AI/ML-driven persona may be among a plurality of AI/ML-driven personas including, but not limited to, at least one of one or more personas based on a fictional literary character, one or more personas based on a non-fictional literary character, one or more personas based on a comic-book character, one or more personas based on a cartoon character, one or more personas based on an anime character, one or more personas based on a manga character, one or more personas based on a television character, one or more personas based on a movie character, one or more personas based on a character from an advertisement, one or more personas based on a mascot, one or more personas based on a meme, one or more personas based on an athlete, one or more personas based on a sports personality, one or more personas based on a news personality, one or more personas based on a political personality, one or more personas based on a reality television personality, one or more personas based on a social media influencer, one or more personas based on a living celebrity, one or more personas based on a deceased celebrity, one or more personas based on a historical figure, one or more personas based on a fictionalization of a historical figure, one or more personas based on a character played by an actor or actress, one or more personas based on a bespoke character, or one or more personas simulating average humans in a geographical area within which the user is currently located or was previously residing, and/or the like.

In some examples, method 500A may continue from the process at block 520a onto the process at block 536 in FIG. 5E following the circular marker denoted, “A.” In some cases, method 500A may continue from the process at block 520a onto the process at block 542 in FIG. 5F following the circular marker denoted, “B.” In some instances, method 500A may continue from the process at block 520a onto the process at block 544 in FIG. 5G following the circular marker denoted, “C.”

In the non-limiting embodiment of FIG. 5B, method 500B, at block 502b, may include causing, by a computing system, at least one AI/ML-driven persona to interact with a user via a UI, the interaction including a conversation between the at least one AI/ML-driven persona and the user. At block 504b, method 500B may include analyzing, by the computing system and using one of at least one AI/ML model, the conversation to identify one or more goals of the conversation related to at least one of one or more products or one or more services provided by a provider. Method 500B may further include, at block 506b, determining, by the computing system and using one of the at least one AI/ML model, a structure of the interaction with the user, based at least in part on the identified one or more goals of the conversation. Method 500B may further include determining, by the computing system and using one of the at least one AI/ML model, one or more first parameters for the determined structure of the interaction with the user, the one or more first parameters defining conversational guardrails for steering the interaction away from conversational tangents and toward achieving at least one goal of the conversation (block 508b). At block 510b, method 500B may include generating, by the computing system and using one of the at least one AI/ML model, one or more first conversational threads configured to achieve at least one goal of the conversation among the one or more goals of the conversation related to the at least one of the one or more products or the one or more services, in some cases, generating the one or more first conversational threads based on the one or more first parameters. Method 500B may further include, at block 512b, causing, by the computing system, the at least one AI/ML-driven persona to continue the conversation with the user using the one or more first conversational threads to work toward achieving the at least one goal of the conversation.

Method 500B, at block 514b, may include, mapping, by the computing system and using one of the at least one AI/ML model, a flow of the interaction with the user. At block 516b, method 500B may include, based on a determination that the flow of the interaction is moving away from achieving the at least one goal of the conversation: determining, by the computing system and using one of the at least one AI/ML model, one or more second parameters for steering the interaction back toward achieving the at least one goal of the conversation. Method 500B may further include, at block 518b, generating, by the computing system and using one of the at least one AI/ML model, one or more second conversational threads configured to steer the interaction back toward achieving the at least one goal of the conversation, in some cases, generating the one or more second conversational threads based on the one or more second parameters. Method 500B may further include causing, by the computing system, the at least one AI/ML-driven persona to continue the conversation with the user using the one or more second conversational threads to steer the interaction back toward achieving the at least one goal of the conversation (block 520b).

According to some embodiments, the one or more goals of the conversion may comprise at least one of purchasing the one or more products, ordering the one or more services, answering one or more questions regarding at least one product among the one or more products, answering one or more questions regarding at least one service among the one or more services, learning how to use at least one product among the one or more products, learning how to use at least one service among the one or more services, troubleshooting at least one issue associated with at least one product among the one or more products, troubleshooting at least one issue associated with at least one service among the one or more services, returning the one or more products, or ending the one or more services, and/or the like.

Merely by way of example, in some cases, the one or more products may comprise one or more of a telephone, a modem, a router, a customer premises equipment (“CPE”), an Ethernet circuit, a network device, a server, a consumer product, an electronic device, sporting goods, office equipment, a home appliance, a media recording device, a media player, a user device, clothing, footwear, a vehicle, or a building, and/or the like. In some examples, the one or more services may comprise one or more of electricity utility service, water utility service, trash and recycling pickup service, telephone service, cellular phone service, satellite telephone service, digital subscriber line (“DSL”) service, Internet service, Ethernet service, optical fiber Internet service, satellite Internet service, streaming media service, downloadable media service, cable television service, or satellite television service, and/or the like.

In some examples, method 500B may continue from the process at block 520b onto the process at block 536 in FIG. 5E following the circular marker denoted, “A.” In some cases, method 500B may continue from the process at block 520b onto the process at block 542 in FIG. 5F following the circular marker denoted, “B.” In some instances, method 500B may continue from the process at block 520b onto the process at block 544 in FIG. 5G following the circular marker denoted, “C.” In some embodiments, method 500B may continue from the process at block 520b onto the process at block 558 in FIG. 5H following the circular marker denoted, “D.”

In the non-limiting embodiment of FIG. 5C, method 500C, at block 502c, may include causing, by a computing system, at least one AI/ML-driven persona to interact with a user via a UI, the interaction including a conversation between the at least one AI/ML-driven persona and the user during which the at least one AI/ML-driven persona engages in assisting the user in learning a skill. In some examples, assisting the user in learning the skill may be based on a lesson plan that, in some cases, may be generated based on instructional data for the skill that may be stored in a database. At block 504c, method 500C may include analyzing, by the computing system and using one of at least one AI/ML model, the interaction to determine a first skill level of the user in the skill, the first skill level including at least one of a level of knowledge of the skill that the user possesses, a level of understanding of the skill by the user, or a level of ability of the user to apply the skill under one or more conditions, and/or the like. Method 500C may further include, at block 506c, determining, by the computing system and using one of the at least one AI/ML model, a structure of the interaction with the user, based at least in part on a first goal of improving the first skill level of the user in the skill. Method 500C may further include determining, by the computing system and using one of the at least one AI/ML model, one or more first parameters for the determined structure of the interaction with the user, the one or more first parameters defining conversational guardrails for steering the interaction away from conversational tangents and toward achieving the first goal (block 508c). Method 500C, at block 522c, may include updating, by the computing system and using one of the at least one AI/ML model, the lesson plan to generate an updated lesson plan, in some cases, based on the analysis of the interaction and/or based on instructional data for the skill.

At block 524c, method 500C may include generating, by the computing system and using one of the at least one AI/ML model, one or more first conversational threads configured to achieve the first goal of improving the first skill level of the user in the skill, based on the analysis of the interaction and based on instructional data for the skill that is accessible from the database, in some cases, generating the one or more first conversational threads may be further based on the one or more first parameters and/or based on the updated lesson plan. Method 500C may further include, at block 526c, causing, by the computing system, the at least one AI/ML-driven persona to continue the conversation with the user using the one or more first conversational threads to work toward achieving the first goal. At block 528c, method 500C may include analyzing, by the computing system and using one of the at least one AI/ML model, the continued conversation to determine effectiveness of the updated lesson plan, in some cases, by determining whether there are any changes to the first skill level of the user in the skill, after use of the one or more first conversational threads. Method 500C may further include, at block 530c, adapting, by the computing system and using one of the at least one AI/ML model, the updated lesson plan to generate an adapted lesson plan, in some cases, based on the analysis of the continued conversation and/or based on the instructional data for the skill. Method 500C may further include generating, by the computing system and using one of the at least one AI/ML model, one or more second conversational threads based on the adapted lesson plan (block 532c); and causing, by the computing system, the at least one AI/ML-driven persona to continue the conversation with the user using the one or more second conversational threads (block 534c).

In some embodiments, the computing system may include, without limitation, at least one of a server, an AI system, a ML system, an AI/ML system, a deep learning (“DL”) system, a user interactive system, a customer interface server, a skill training system, an automated tutoring system, an education server, an education facility computing system, a cloud computing system, or a distributed computing system, and/or the like.

According to some embodiments, the skill may be among a plurality of skills including, but not limited to, at least one of skill in a language among a plurality of languages (including, without limitation, English, Mandarin, Hindi, Spanish, French, Arabic, Bengali, Russian, Portuguese, Indonesian, etc.), skill in understanding a culture among a plurality of cultures, skill in living in nature, skill in living in small population centers, skill in living in large population centers, skill in living in rural areas, skill in living in cities, skill in navigating tourist areas, skill in an elementary school course, skill in a middle school course, skill in a high school course, skill in a college course, skill in a university course, skill in a vocational course, skill in mathematics, skill in physics, skill in chemistry, skill in biology, skill in sociology, skill in philosophy, skill in psychology, skill in computer programming, skill in literary studies, skill in linguistics, skill in writing, skill in civics or social studies, skill in historical studies, skill in geography, skill in geology, skill in engineering, skill in educating others, skill in training animals, skill in home repairs, skill in vehicle repairs, skill in appliance repairs, skill in computer repairs, skill in mobile device repairs, skill in assembling a consumer product, skill in construction, skill in driving, skill in sports activity, skill in recreational activity, skill in card games, skill in board games, skill in trivia games, skill in cooking, skill in butchering meats, skill in cleaning, skill in crafting, skill in carpentry, skill in metalworking, skill in smithing, skill in artistic forms, skill in painting, skill in first aid, skill in navigation, skill in buying, skill in selling, skill in bartering, skill in negotiation, skill in trading, skill in marketing, skill in accounting, skill in business development, skill in public speaking, skill in giving presentations, skill in communication, skill in management, or skill in leadership, and/or the like.

In some examples, method 500C may continue from the process at block 534c onto the process at block 536 in FIG. 5E following the circular marker denoted, “A.” In some cases, method 500C may continue from the process at block 534c onto the process at block 542 in FIG. 5F following the circular marker denoted, “B.” In some instances, method 500C may continue from the process at block 534c onto the process at block 544 in FIG. 5G following the circular marker denoted, “C.” In some embodiments, method 500C may continue from the process at block 534c onto the process at block 570 in FIG. 5I following the circular marker denoted, “E.”

In the non-limiting embodiment of FIG. 5D, method 500D, at block 502d, may include causing, by a computing system, at least one AI/ML-driven persona to interact with a user via a UI, the interaction including a conversation between the at least one AI/ML-driven persona and the user during which the at least one AI/ML-driven persona engages in assisting the user in learning one or more social skills to interact with other people. In some examples, assisting the user in learning the one or more social skills may be based on a therapy plan that, in some cases, may be generated based on social or behavioral therapy data for the at least one set of social skills that may be stored in a database. At block 504d, method 500D may include analyzing, by the computing system and using one of at least one AI/ML model, the interaction to determine a first skill level of the user in at least one set of social skills among the one or more social skills, the first skill level comprising a level of competence in the at least one set of social skill that the user possesses. Method 500D may further include, at block 506d, determining, by the computing system and using one of the at least one AI/ML model, a structure of the interaction with the user, based at least in part on a first goal of developing the first skill level of the user in the at least one set of social skill. Method 500D may further include determining, by the computing system and using one of the at least one AI/ML model, one or more first parameters for the determined structure of the interaction with the user, the one or more first parameters defining conversational guardrails for steering the interaction away from conversational tangents and toward achieving the first goal (block 508d). Method 500D, at block 522d, may include updating, by the computing system and using one of the at least one AI/ML model, the therapy plan to generate an updated therapy plan, in some cases, based on the analysis of the interaction and/or based on social or behavioral therapy data for the at least one set of social skills.

At block 524d, method 500D may include generating, by the computing system and using one of the at least one AI/ML model, one or more first conversational threads configured to achieve the first goal of developing the first skill level of the user in the at least one set of social skill, based on the analysis of the interaction and based on social or behavioral therapy data for the at least one set of social skills that is accessible from the database, in some cases, generating the one or more first conversational threads may be further based on the one or more first parameters and/or based on the updated therapy plan. Method 500D may further include, at block 526d, causing, by the computing system, the at least one AI/ML-driven persona to continue the conversation with the user using the one or more first conversational threads to work toward achieving the first goal. At block 528d, method 500D may include analyzing, by the computing system and using one of the at least one AI/ML model, the continued conversation to determine effectiveness of the updated therapy plan, by determining whether there are any changes to the first skill level of the user in the at least one set of social skills, after use of the one or more first conversational threads. Method 500D may further include, at block 530d, adapting, by the computing system and using one of the at least one AI/ML model, the updated therapy plan to generate an adapted therapy plan, in some cases, based on the analysis of the continued conversation and/or based on the social or behavioral therapy data for the at least one set of social skills. Method 500D may further include generating, by the computing system and using one of the at least one AI/ML model, one or more second conversational threads based on the adapted therapy plan (block 532d); and causing, by the computing system, the at least one AI/ML-driven persona to continue the conversation with the user using the one or more second conversational threads (block 534d).

In some embodiments, the computing system may include, without limitation, at least one of a server, an AI system, a ML system, an AI/ML system, a deep learning (“DL”) system, a user interactive system, a customer interface server, a social skills training system, a behavioral skills training system, a behavioral therapy system, an education server, an education facility computing system, a cloud computing system, or a distributed computing system, and/or the like.

According to some embodiments, the one or more social skills may comprise at least one of social coordination skills, mentoring skills, negotiation skills, persuasion skills, psychosocial service orientation skills, social perceptiveness skills, active listening skills, delegation skills, decision-making skills, problem-solving skills, creative thinking skills, critical thinking skills, communication skills, interpersonal skills, self-awareness skills, empathy skills, assertiveness skills, equanimity skills, psychological resilience skills, or coping skills, and/or the like.

In some examples, method 500D may continue from the process at block 534d onto the process at block 536 in FIG. 5E following the circular marker denoted, “A.” In some cases, method 500D may continue from the process at block 534d onto the process at block 542 in FIG. 5F following the circular marker denoted, “B.” In some instances, method 500D may continue from the process at block 534d onto the process at block 544 in FIG. 5G following the circular marker denoted, “C.” In some embodiments, method 500D may continue from the process at block 534d onto the process at block 570 in FIG. 5I following the circular marker denoted, “E.”

At block 536 in FIG. 5E (following the circular marker denoted, “A,” in FIG. 5A, FIG. 5B, FIG. 5C, or FIG. 5D), method 500 may include analyzing, by the computing system and using one of the at least one AI/ML model, the interaction to identify one or more observable characteristics of the user. In some embodiments, the one or more observable characteristics may include (where applicable), but are not limited to, at least one of one or more speech patterns of the user, a language used by the user, whether the user has an accent, what accent the user has, whether English is a second language for the user, one or more non-verbal cues of the user, a demeanor of the user, a sentiment of the user, or an emotional state of the user, and/or the like.

Method 500 may further include, at block 538, accessing and analyzing, by the computing system and using one of the at least one AI/ML model, stored information associated with the user to identify one or more conversation points. In some embodiments, the stored information may include (where applicable), but is not limited to, at least one of account information associated with the user, contact information associated with the user, billing information associated with the user, one or more contracts between the provider and the user, order history data associated with the user, previous interactions with the user, previous trouble tickets associated with the user, historical data associated with the user, demographic information about the user, personal information about the user, user-volunteered information regarding general interests of the user, information regarding a market segment within which the user is classified, or societal information for a societal segment to which the user belongs, and/or the like. Method 500 may further include causing, by the computing system, the at least one AI/ML-driven persona to adapt by modifying the interaction with the user, based at least in part on at least one of the identified one or more observable characteristics of the user or the identified one or more conversation points, to enhance or improve the interaction with the user (block 540).

At block 542 in FIG. 5F (following the circular marker denoted, “B,” in FIG. 5A, FIG. 5B, FIG. 5C, or FIG. 5D), method 500 may include identifying and verifying, by the computing system, an identity of the user, in some cases, based at least in part on one or more of caller identification (“ID”) information, the account information associated with the user, a voiceprint of the user, two-factor authentication, or information provided by the user, and/or the like.

In some embodiments, the UI may be a visual-based UI. At block 544 in FIG. 5G (following the circular marker denoted, “C,” in FIG. 5A, FIG. 5B, FIG. 5C, or FIG. 5D), method 500 may include, concurrent with the interaction with the user, generating, by the computing system, an avatar for each of the at least one AI/ML-driven persona. Method 500 may further include, at block 546, displaying, by the computing system and within the UI, the avatar for each of the at least one AI/ML-driven persona. Method 500 may further include animating, by the computing system and within the UI, the avatar in synchronization with the conversation with the user (block 548), where the interaction may further include the animation of the avatar. Method 500 may continue onto the process at block 550 and/or the process at block 554. In some instances, each AI/ML-driven persona may have a set personality, the set personality including at least one of a set speech pattern, a set mannerism, a set command of one or more languages, a set accent, a set collection of non-verbal cues, or a set collection of emotional demeanors, and/or the like. In some examples, each of the interaction, the conversation, the one or more first conversational threads, and the animation of each AI/ML-driven persona may be performed in a manner consistent with the set personality of said AI/ML-driven persona.

In some cases, method 500 may further include, in response to user selection of a gamification mode, performing the following: generating, by the computing system, a visual representation of a list of the one or more goals (block 550); and in response to achieving a goal among the one or more goals, generating, by the computing system, an animation of one or more of the at least one AI/ML-driven persona performing one or more actions to mark achievement of the goal, the one or more actions being performed in a manner consistent with the set personality of each of the one or more of the at least one AI/ML-driven persona (block 552). In some instances, one or more actions may include, but are not limited to, checking off the achieved goal, removing the achieved goal from the list, or marking the achieved goal as having been achieved, and/or the like.

In some examples, method 500 may further include (although not shown in FIG. 5), prior to the conversation or during a setup phase, performing one of: (a) receiving, by the computing system, a user selection of the at least one AI/ML-driven persona from a licensed set of AI/ML-driven personas among the plurality of AI/ML-driven personas with whom to interact; (b) identifying, by the computing system, the user, and selecting, by the computing system, the at least one AI/ML-driven persona from the licensed set of AI/ML-driven personas to match the user for interacting with the user, based on information regarding the identified user; (c) setting, by the computing system, a default set of AI/ML-driven personas from the licensed set of AI/ML-driven personas, wherein the default set of AI/ML-driven personas includes the at least one AI/ML-driven persona; or (d) randomly selecting, by the computing system, the at least one AI/ML-driven persona from the licensed set of AI/ML-driven personas for interacting with the user; or the like.

In some instances, method 500 may further include (where applicable) at least one of: (1) adapting or adjusting, by the computing system and using one of the at least one AI/ML model, a personality of one or more of the at least one AI/ML-driven persona to mold to or match a determined personality of the user, wherein the personality of the user may be determined based on at least one of analysis of the interaction with the user, analysis of a previous interaction with the user, or known information about the user, and/or the like (block 554); (2) adapting or adjusting, by the computing system and using one of the at least one AI/ML model, one or more interaction characteristics of one or more of the at least one AI/ML-driven persona to match to a determined corresponding interaction characteristic of the user, the one or more interaction characteristics including at least one of speech pattern, language, accent, cultural mannerisms, cultural phraseology, general mannerisms, general phraseology, slang, jargon, or sentiment, and/or the like (block 556); (3) adapting or adjusting, by the computing system and using one of the at least one AI/ML model, a personality of one or more of the at least one AI/ML-driven persona to mold to or match a personality of a person whom the user is determined to respect or feel comfortable talking with, wherein the personality of the person whom the user is determined to respect or feel comfortable talking with is determined based on at least one of analysis of the interaction with the user, analysis of a previous interaction with the user, or known information about the user, and/or the like (not shown); or (4) adapting or adjusting, by the computing system and using one of the at least one AI/ML model, one or more interaction characteristics of one or more of the at least one AI/ML-driven persona to match to a determined corresponding interaction characteristic of the person whom the user is determined to respect or feel comfortable talking with, the one or more interaction characteristics including at least one of speech pattern, language, accent, cultural mannerisms, cultural phraseology, general mannerisms, general phraseology, slang, jargon, or sentiment, and/or the like (not shown); or the like.

At block 558 in FIG. 5H (following the circular marker denoted, “D,” in FIG. 5B), method 500B may include, concurrent with the interaction with the user, causing, by the computing system, the at least one AI/ML-driven persona to investigate a status of the at least one of the one or more products or the one or more services. Method 500B may further include, at block 560, determining, by the computing system, whether the computing system is capable of remotely addressing one or more issues with the at least one of the one or more products or the one or more services. Method 500B may further include, based on a determination that the computing system is capable of remotely addressing one or more issues with the at least one of the one or more products or the one or more services, generating, by the computing system, a conversational message indicating that the at least one AI/ML-driven persona is able to remotely address the one or more issues with the at least one of the one or more products or the one or more services and will proceed to do so (block 562), and initiating, by the computing system, one or more processes to remotely address the one or more issues with the at least one of the one or more products or the one or more services (block 564). In some examples, determining whether the computing system is capable of remotely addressing the one or more issues with the at least one of the one or more products or the one or more services (at block 560) may include remotely accessing, by the computing system, one or more systems associated with the at least one of the one or more products or the one or more services (block 566), and remotely running, by the computing system, one or more tests on the one or more systems, the one or more tests including a connectivity and control test (block 568).

At block 570 in FIG. 5I (following the circular marker denoted, “E,” in FIG. 5C or FIG. 5D), method 500C or method 500D may include mapping, by the computing system and using one of the at least one AI/ML model, a flow of the interaction with the user. Method 500C or method 500D may further include, based on a determination that the flow of the interaction is moving away from achieving the first goal: determining, by the computing system and using one of the at least one AI/ML model, one or more third parameters for steering the interaction back toward achieving the first goal (block 572); generating, by the computing system and using one of the at least one AI/ML model, one or more third conversational threads configured to steer the interaction back toward achieving the first goal, in some cases, based on the one or more third parameters (block 574); and causing, by the computing system, the at least one AI/ML-driven persona to continue the conversation with the user using the one or more third conversational threads (block 576).

### Exemplary System and Hardware Implementation

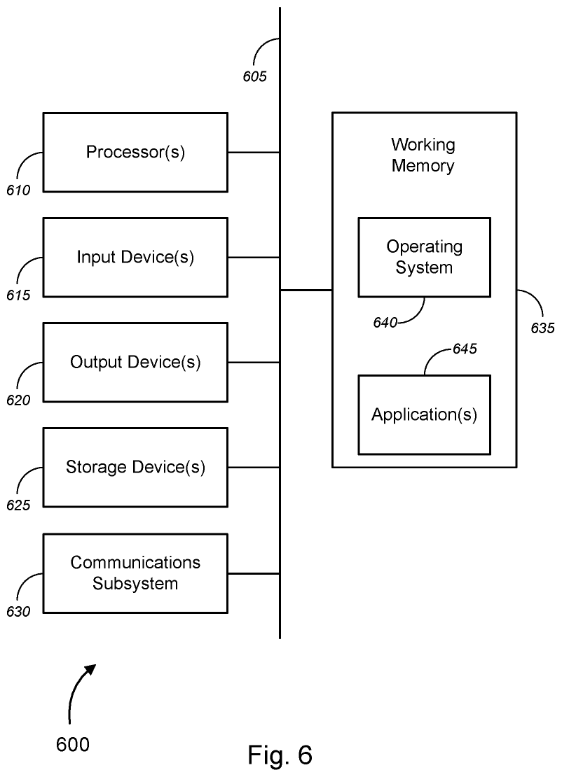

FIG. 6 is a block diagram illustrating an exemplary computer or system hardware architecture, in accordance with various embodiments. FIG. 6 provides a schematic illustration of one embodiment of a computer system 600 of the service provider system hardware that can perform the methods provided by various other embodiments, as described herein, and/or can perform the functions of computer or hardware system (i.e., computing systems 105, 105a, and 105b, user interactive systems 115a and 115b, artificial intelligence (“AI”)/machine learning (“ML”) systems 120a and 120b, user device 140, gateway device 160, provider server(s) 175a, training server(s) 190a, and therapy server(s) 195a, etc.), as described above. It should be noted that FIG. 6 is meant only to provide a generalized illustration of various components, of which one or more (or none) of each may be utilized as appropriate. FIG. 6, therefore, broadly illustrates how individual system elements may be implemented in a relatively separated or relatively more integrated manner.

The computer or hardware system 600—which might represent an embodiment of the computer or hardware system (i.e., computing systems 105, 105a, and 105b, user interactive systems 115a and 115b, AI/ML systems 120a and 120b, user device 140, gateway device 160, provider server(s) 175a, training server(s) 190a, and therapy server(s) 195a, etc.), described above with respect to FIGS. 1-5—is shown comprising hardware elements that can be electrically coupled via a bus 605 (or may otherwise be in communication, as appropriate). The hardware elements may include one or more processors 610, including, without limitation, one or more general-purpose processors and/or one or more special-purpose processors (such as microprocessors, digital signal processing chips, graphics acceleration processors, and/or the like); one or more input devices 615, which can include, without limitation, a mouse, a keyboard, and/or the like; and one or more output devices 620, which can include, without limitation, a display device, a printer, and/or the like.

The computer or hardware system 600 may further include (and/or be in communication with) one or more storage devices 625, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer or hardware system 600 might also include a communications subsystem 630, which can include, without limitation, a modem, a network card (wireless or wired), an infra-red communication device, a wireless communication device and/or chipset (such as a Bluetooth™ device, an 802.11 device, a Wi-Fi device, a WiMAX device, a wireless wide area network (“WWAN”) device, cellular communication facilities, etc.), and/or the like. The communications subsystem 630 may permit data to be exchanged with a network (such as the network described below, to name one example), with other computer or hardware systems, and/or with any other devices described herein. In many embodiments, the computer or hardware system 600 will further comprise a working memory 635, which can include a RAM or ROM device, as described above.

The computer or hardware system 600 also may comprise software elements, shown as being currently located within the working memory 635, including an operating system 640, device drivers, executable libraries, and/or other code, such as one or more application programs 645, which may comprise computer programs provided by various embodiments (including, without limitation, hypervisors, virtual machines (“VMs”), and the like), and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be encoded and/or stored on a non-transitory computer readable storage medium, such as the storage device(s) 625 described above. In some cases, the storage medium might be incorporated within a computer system, such as the system 600. In other embodiments, the storage medium might be separate from a computer system (i.e., a removable medium, such as a compact disc, etc.), and/or provided in an installation package, such that the storage medium can be used to program, configure, and/or adapt a general purpose computer with the instructions/code stored thereon. These instructions might take the form of executable code, which is executable by the computer or hardware system 600 and/or might take the form of source and/or installable code, which, upon compilation and/or installation on the computer or hardware system 600 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, field-programmable gate arrays, application-specific integrated circuits, and/or the like) might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer or hardware system (such as the computer or hardware system 600) to perform methods in accordance with various embodiments of the invention. According to a set of embodiments, some or all of the procedures of such methods are performed by the computer or hardware system 600 in response to processor 610 executing one or more sequences of one or more instructions (which might be incorporated into the operating system 640 and/or other code, such as an application program 645) contained in the working memory 635. Such instructions may be read into the working memory 635 from another computer readable medium, such as one or more of the storage device(s) 625. Merely by way of example, execution of the sequences of instructions contained in the working memory 635 might cause the processor(s) 610 to perform one or more procedures of the methods described herein.

The terms “machine readable medium” and “computer readable medium,” as used herein, refer to any medium that participates in providing data that causes a machine to operate in a specific fashion. In an embodiment implemented using the computer or hardware system 600, various computer readable media might be involved in providing instructions/code to processor(s) 610 for execution and/or might be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 625. Volatile media includes, without limitation, dynamic memory, such as the working memory 635. In some alternative embodiments, a computer readable medium may take the form of transmission media, which includes, without limitation, coaxial cables, copper wire, and fiber optics, including the wires that comprise the bus 605, as well as the various components of the communication subsystem 630 (and/or the media by which the communications subsystem 630 provides communication with other devices). In an alternative set of embodiments, transmission media can also take the form of waves (including without limitation radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 610 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer or hardware system 600. These signals, which might be in the form of electromagnetic signals, acoustic signals, optical signals, and/or the like, are all examples of carrier waves on which instructions can be encoded, in accordance with various embodiments of the invention.

The communications subsystem 630 (and/or components thereof) generally will receive the signals, and the bus 605 then might carry the signals (and/or the data, instructions, etc. carried by the signals) to the working memory 635, from which the processor(s) 605 retrieves and executes the instructions. The instructions received by the working memory 635 may optionally be stored on a storage device 625 either before or after execution by the processor(s) 610.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to particular structural and/or functional components for ease of description, methods provided by various embodiments are not limited to any particular structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in a particular order for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a particular structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A method, comprising:
causing, by a computing system, at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning a skill;
analyzing, by the computing system and using one of at least one AI/ML model, the interaction to determine a first skill level of the user in the skill, the first skill level comprising at least one of a level of knowledge of the skill that the user possesses, a level of understanding of the skill by the user, or a level of ability of the user to apply the skill under one or more conditions;
generating, by the computing system and using one of the at least one AI/ML model, one or more first conversational threads configured to achieve a first goal of improving the first skill level of the user in the skill, based on the analysis of the interaction and based on instructional data for the skill that is accessible from a database; and
causing, by the computing system, the at least one AI/ML-driven persona to continue the conversation with the user using the one or more first conversational threads to work toward achieving the first goal.

2. The method of claim 1, wherein the computing system comprises at least one of a server, an AI system, a ML system, an AI/ML system, a deep learning (“DL”) system, a user interactive system, a customer interface server, a skill training system, an automated tutoring system, an education server, an education facility computing system, a cloud computing system, or a distributed computing system, wherein the UI comprises one of a voice-only UI, a telephone communication UI, a video-only UI, a video with voice UI, a chat UI, a software application (“app”) UI, a holographic UI, a virtual reality (“VR”)-based UI, an augmented reality (“AR”)-based UI, a mixed reality (“MR”)-based UI, or a web-portal-based UI.

3. The method of claim 1, wherein the skill is among a plurality of skills comprising at least one of skill in a language among a plurality of languages, skill in understanding a culture among a plurality of cultures, skill in living in nature, skill in living in small population centers, skill in living in large population centers, skill in living in rural areas, skill in living in cities, skill in navigating tourist areas, skill in an elementary school course, skill in a middle school course, skill in a high school course, skill in a college course, skill in a university course, skill in a vocational course, skill in mathematics, skill in physics, skill in chemistry, skill in biology, skill in sociology, skill in philosophy, skill in psychology, skill in computer programming, skill in literary studies, skill in linguistics, skill in writing, skill in civics or social studies, skill in historical studies, skill in geography, skill in geology, skill in engineering, skill in educating others, skill in training animals, skill in home repairs, skill in vehicle repairs, skill in appliance repairs, skill in computer repairs, skill in mobile device repairs, skill in assembling a consumer product, skill in construction, skill in driving, skill in sports activity, skill in recreational activity, skill in card games, skill in board games, skill in trivia games, skill in cooking, skill in butchering meats, skill in cleaning, skill in crafting, skill in carpentry, skill in metalworking, skill in smithing, skill in artistic forms, skill in painting, skill in first aid, skill in navigation, skill in buying, skill in selling, skill in bartering, skill in negotiation, skill in trading, skill in marketing, skill in accounting, skill in business development, skill in public speaking, skill in giving presentations, skill in communication, skill in management, or skill in leadership.

4. The method of claim 3, further comprising at least one of:
using a first set of AI/ML-driven personas to assist the user in learning a first skill among the plurality of skills; and using a second set of AI/ML-driven personas that is different from the first set of AI/ML-driven personas to assist the user in learning a second skill among the plurality of skills that is different from the first skill; or
using a first set of learning strategies among a plurality of learning strategies for assisting the user in learning the first skill; and using a second set of learning strategies among the plurality of learning strategies that is different from the first set of learning strategies for assisting the user in learning the second skill, wherein the plurality of learning strategies includes rote learning, learning using flash cards or virtual flash cards, learning by interaction, learning by practice, auditory learning, visual learning, or learning using a combination of two or more of said learning strategies.

5. The method of claim 1, wherein assisting the user in learning the skill is based on a lesson plan that is generated based on the instructional data for the skill, wherein the method further comprises:
updating, by the computing system and using one of the at least one AI/ML model, the lesson plan to generate an updated lesson plan, based on the analysis of the interaction and based on instructional data for the skill, wherein the one or more first conversational threads are further based on the updated lesson plan;
analyzing, by the computing system and using one of the at least one AI/ML model, the continued conversation to determine effectiveness of the updated lesson plan, by determining whether there are any changes to the first skill level of the user in the skill, after use of the one or more first conversational threads;
adapting, by the computing system and using one of the at least one AI/ML model, the updated lesson plan to generate an adapted lesson plan, based on the analysis of the continued conversation and based on the instructional data for the skill;
generating, by the computing system and using one of the at least one AI/ML model, one or more second conversational threads based on the adapted lesson plan; and
causing, by the computing system, the at least one AI/ML-driven persona to continue the conversation with the user using the one or more second conversational threads.

6. The method of claim 5, wherein the skill is skill in a first language among a plurality of languages, wherein analyzing the conversation or the continued conversation comprises at least one of:
analyzing, by the computing system and using one of at least one AI/ML model, the interaction to determine at least one of a breadth of a breadth of listening vocabulary, a depth of listening vocabulary, a breadth of speaking vocabulary, a depth of speaking vocabulary, reading vocabulary, a depth of reading vocabulary, a breadth of writing vocabulary, or a depth of writing vocabulary that the user has in the first language;
analyzing, by the computing system and using one of at least one AI/ML model, the interaction to determine the user's proficiency and understanding of grammar in the first language, the grammar including at least one of tense, number, noun classes, locative relations, syntax, grammatical structure, or grammatical gender;
analyzing, by the computing system and using one of at least one AI/ML model, the interaction to determine the user's pronunciation of words in the first language;
analyzing, by the computing system and using one of at least one AI/ML model, the interaction to determine an optimal learning strategy among a plurality of learning strategies for the user to learn the first language, the plurality of learning strategies including learning by flash cards or virtual flash cards, learning by usage in sentences, learning by vocabulary drills, learning by exposure to particular words in different contexts, learning by language immersion, learning by interaction, learning by reading sentences or passages, learning by speaking sentences, learning by listening to conversations, auditory learning, visual learning, learning by translating to a second language among the plurality of languages that is different from the first language, or learning using a combination of two or more of said learning strategies;
analyzing, by the computing system and using one of at least one AI/ML model, the interaction to determine how quickly the user interacts or responds; or
analyzing, by the computing system and using one of at least one AI/ML model, the interaction to determine which AI/ML-driven persona or which combination of two or more AI/ML-driven personas best assists the user in learning the first language.

7. The method of claim 5, wherein updating the lesson plan or adapting the updated lesson plan comprises at least one of:
changing, by the computing system, one or more first AI/ML-driven personas among the at least one AI/ML-driven persona to one or more second AI/ML-driven persona among the at least one AI/ML-driven persona;
changing, by the computing system, at least one of a voice or speech pattern, a tone, an accent, a pitch, a cadence, or a gender of the at least one AI/ML-driven persona;
changing, by the computing system, at least one of a time of day, a time of week, or a time of month that the lesson plan or the updated lesson plan is implemented;
changing, by the computing system, a pace of the lesson plan or the updated lesson plan; or
changing, by the computing system, a learning strategy among a plurality of learning strategies from a first learning strategy to a second learning strategy, the plurality of learning strategies including rote learning, learning using flash cards or virtual flash cards, learning by interaction, learning by practice, auditory learning, visual learning, or learning using a combination of two or more of said learning strategies.

8. The method of claim 1, further comprising:
analyzing, by the computing system and using one of the at least one AI/ML model, the interaction to identify one or more observable characteristics of the user, the one or more observable characteristics including at least one of one or more speech patterns of the user, a language used by the user, whether the user has an accent, what accent the user has, one or more non-verbal cues of the user, a demeanor of the user, a sentiment of the user, or an emotional state of the user;
accessing and analyzing, by the computing system and using one of the at least one AI/ML model, stored information associated with the user to identify one or more conversation points, the stored information including at least one of account information associated with the user, contact information associated with the user, order history data associated with the user, previous interactions with the user, historical data associated with the user, demographic information about the user, personal information about the user, user-volunteered information regarding general interests of the user, information regarding a market segment within which the user is classified, or societal information for a societal segment to which the user belongs; and
causing, by the computing system, the at least one AI/ML-driven persona to adapt by modifying the interaction with the user, based at least in part on at least one of the identified one or more observable characteristics of the user or the identified one or more conversation points, to enhance or improve the interaction with the user.

9. The method of claim 1, wherein the at least one AI/ML-driven persona is among a plurality of AI/ML-driven personas comprising at least one of one or more personas based on a fictional literary character, one or more personas based on a non-fictional literary character, one or more personas based on a comic-book character, one or more personas based on a cartoon character, one or more personas based on an anime character, one or more personas based on a manga character, one or more personas based on a television character, one or more personas based on a movie character, one or more personas based on a character from an advertisement, one or more personas based on a mascot, one or more personas based on a meme, one or more personas based on an athlete, one or more personas based on a sports personality, one or more personas based on a news personality, one or more personas based on a political personality, one or more personas based on a reality television personality, one or more personas based on a social media influencer, one or more personas based on a living celebrity, one or more personas based on a deceased celebrity, one or more personas based on a historical figure, one or more personas based on a fictionalization of a historical figure, one or more personas based on a character played by an actor or actress, one or more personas based on a bespoke character, or one or more personas simulating average humans in a geographical area within which the user is currently located or was previously residing.

10. The method of claim 9, wherein the UI is a visual-based UI, wherein the method further comprises:
generating, by the computing system, an avatar for each of the at least one AI/ML-driven persona;
displaying, by the computing system and within the UI, the avatar for each of the at least one AI/ML-driven persona; and
animating, by the computing system and within the UI, the avatar in synchronization with the conversation with the user, wherein the interaction further comprises the animation of the avatar.

11. The method of claim 10, wherein each AI/ML-driven persona has a set personality, the set personality including at least one of a set speech pattern, a set mannerism, a set command of one or more languages, a set accent, a set collection of non-verbal cues, or a set collection of emotional demeanors, wherein each of the interaction, the conversation, the one or more first conversational threads, and the animation of each AI/ML-driven persona is performed in a manner consistent with the set personality of said AI/ML-driven persona.

12. The method of claim 10, further comprising, in response to user selection of a gamification mode, performing the following:
generating, by the computing system, a visual representation of a list of one or more goals for learning the skill, the one or more goals for learning the skill including the first goal; and
in response to achieving a goal among the one or more goals for learning the skill, generating, by the computing system, an animation of one or more of the at least one AI/ML-driven persona performing one or more actions comprising checking off the achieved goal, removing the achieved goal from the list, or marking the achieved goal as having been achieved, the one or more actions being performed in a manner consistent with the set personality of each of the one or more of the at least one AI/ML-driven persona.

13. The method of claim 10, further comprising, prior to the conversation or during a setup phase, performing one of:
receiving, by the computing system, a user selection of the at least one AI/ML-driven persona from a licensed set of AI/ML-driven personas among the plurality of AI/ML-driven personas with whom to interact;
identifying, by the computing system, the user, and selecting, by the computing system, the at least one AI/ML-driven persona from the licensed set of AI/ML-driven personas to match the user for interacting with the user, based on information regarding the identified user;
setting, by the computing system, a default set of AI/ML-driven personas from the licensed set of AI/ML-driven personas, wherein the default set of AI/ML-driven personas comprises the at least one AI/ML-driven persona; or
randomly selecting, by the computing system, the at least one AI/ML-driven persona from the licensed set of AI/ML-driven personas for interacting with the user.

14. The method of claim 10, further comprising at least one of:
adapting or adjusting, by the computing system and using one of the at least one AI/ML model, a personality of one or more of the at least one AI/ML-driven persona to mold to or match a determined personality of the user, wherein the personality of the user is determined based on at least one of analysis of the interaction with the user, analysis of a previous interaction with the user, or known information about the user; or
adapting or adjusting, by the computing system and using one of the at least one AI/ML model, one or more interaction characteristics of one or more of the at least one AI/ML-driven persona to match to a determined corresponding interaction characteristic of the user, the one or more interaction characteristics including at least one of speech pattern, language, accent, cultural mannerisms, cultural phraseology, general mannerisms, general phraseology, slang, jargon, or sentiment.

15. The method of claim 1, further comprising:
determining, by the computing system and using one of the at least one AI/ML model, a structure of the interaction with the user, based at least in part on the first goal; and
determining, by the computing system and using one of the at least one AI/ML model, one or more first parameters for the determined structure of the interaction with the user, the one or more first parameters defining conversational guardrails for steering the interaction away from conversational tangents and toward achieving the first goal;
wherein the one or more first conversational threads are generated based on the one or more first parameters.

16. The method of claim 15, further comprising:
mapping, by the computing system and using one of the at least one AI/ML model, a flow of the interaction with the user; and
based on a determination that the flow of the interaction is moving away from achieving the first goal:
determining, by the computing system and using one of the at least one AI/ML model, one or more third parameters for steering the interaction back toward achieving the first goal;
generating, by the computing system and using one of the at least one AI/ML model, one or more third conversational threads configured to steer the interaction back toward achieving the first goal, based on the one or more third parameters; and
causing, by the computing system, the at least one AI/ML-driven persona to continue the conversation with the user using the one or more third conversational threads.

17. A system, comprising:
a computing system, comprising:
at least one first processor; and
a first non-transitory computer readable medium communicatively coupled to the at least one first processor, the first non-transitory computer readable medium having stored thereon computer software comprising a first set of instructions that, when executed by the at least one first processor, causes the computing system to:
cause at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning a skill;
analyze, using one of at least one AI/ML model, the interaction to determine a first skill level of the user in the skill, the first skill level comprising at least one of a level of knowledge of the skill that the user possesses, a level of understanding of the skill by the user, or a level of ability of the user to apply the skill under one or more conditions;
generate, using one of the at least one AI/ML model, one or more first conversational threads configured to achieve a first goal of improving the first skill level of the user in the skill, based on the analysis of the interaction and based on instructional data for the skill that is accessible from a database; and
cause the at least one AI/ML-driven persona to continue the conversation with the user using the one or more second conversational threads to work toward achieving the first goal.

18. A method, comprising:
causing, by a computing system, at least one artificial intelligence (“AI”)/machine learning (“ML”)-driven persona to interact with a user via a user interface (“UI”), the interaction including a conversation between the at least one AI/ML-driven persona and the user during which the at least one AI/ML-driven persona engages in assisting the user in learning a first language among a plurality of languages;
analyzing, by the computing system and using one of at least one AI/ML model, the interaction to determine a first skill level of the user in the first language, the first skill level comprising at least one of a level of knowledge of the first language that the user possesses, a level of understanding of the first language by the user, or a level of ability of the user to apply the first language under one or more conditions;
generating, by the computing system and using one of the at least one AI/ML model, one or more first conversational threads configured to achieve a first goal of improving the first skill level of the user in the first language, based on the analysis of the interaction and based on instructional data for the first language that is accessible from a database; and
causing, by the computing system, the at least one AI/ML-driven persona to continue the conversation with the user using the one or more first conversational threads to work toward achieving the first goal.

19. The method of claim 18, wherein assisting the user in learning the first language is based on a lesson plan that is generated based on the instructional data for the first language, wherein the method further comprises:
updating, by the computing system and using one of the at least one AI/ML model, the lesson plan to generate an updated lesson plan, based on the analysis of the interaction and based on instructional data for the first language, wherein the one or more first conversational threads are further based on the updated lesson plan;
analyzing, by the computing system and using one of the at least one AI/ML model, the continued conversation to determine effectiveness of the updated lesson plan, by determining whether there are any changes to the first skill level of the user in the first language the first language the first language the first language, after use of the one or more first conversational threads;
adapting, by the computing system and using one of the at least one AI/ML model, the updated lesson plan to generate an adapted lesson plan, based on the analysis of the continued conversation and based on the instructional data for the first language;
generating, by the computing system and using one of the at least one AI/ML model, one or more second conversational threads based on the adapted lesson plan; and
causing, by the computing system, the at least one AI/ML-driven persona to continue the conversation with the user using the one or more second conversational threads.

20. The method of claim 19, wherein analyzing the conversation or the continued conversation comprises at least one of:
analyzing, by the computing system and using one of at least one AI/ML model, the interaction to determine at least one of a breadth of a breadth of listening vocabulary, a depth of listening vocabulary, a breadth of speaking vocabulary, a depth of speaking vocabulary, reading vocabulary, a depth of reading vocabulary, a breadth of writing 6 vocabulary, or a depth of writing vocabulary that the user has in the first language;
analyzing, by the computing system and using one of at least one AI/ML model, the interaction to determine the user's proficiency and understanding of grammar in the first language, the grammar including at least one of tense, number, noun classes, locative relations, syntax, grammatical structure, or grammatical gender;
analyzing, by the computing system and using one of at least one AI/ML model, the interaction to determine the user's pronunciation of words in the first language;
analyzing, by the computing system and using one of at least one AI/ML model, the interaction to determine an optimal learning strategy among a plurality of learning strategies for the user to learn the first language, the plurality of learning strategies including learning by flash cards or virtual flash cards, learning by usage in sentences, learning by vocabulary drills, learning by exposure to particular words in different contexts, learning by language immersion, learning by interaction, learning by reading sentences or passages, learning by speaking sentences, learning by listening to conversations, auditory learning, visual learning, learning by translating to a second language among the plurality of languages that is different from the first language, or learning using a combination of two or more of said learning strategies;
analyzing, by the computing system and using one of at least one AI/ML model, the interaction to determine how quickly the user interacts or responds; or
analyzing, by the computing system and using one of at least one AI/ML model, the interaction to determine which AI/ML-driven persona or which combination of two or more AI/ML-driven personas best assists the user in learning the first language.
